(19) United States
(12) Patent Application Publication
(10) Pub. No.: US 2025/0160666 A1
(43) Pub. Date: May 22, 2025

(54) DEVICE, SYSTEM, AND METHOD FOR BLOOD PRESSURE MEASUREMENT

(71) Applicant: UNIVERSITY OF PITTSBURGH - OF THE COMMONWEALTH SYSTEM OF HIGHER EDUCATION, Pittsburgh, PA (US)

(72) Inventors: Ramakrishna MUKKAMALA, Sewickley, PA (US); Mark A. FREITHALER, Pittsburgh, PA (US); Sanjeev Govinddas SHROFF, Pittsburgh, PA (US); Vishaal DHAMOTHARAN, Pittsburgh, PA (US); Anand CHANDRASEKHAR, Medford, MA (US); Cederick LANDRY, Sherbrooke, CA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 18/987,939

(22) Filed: Dec. 19, 2024

Related U.S. Application Data
(63) Continuation of application No. PCT/US23/27390, filed on Jul. 11, 2023.
(60) Provisional application No. 63/388,021, filed on Jul. 11, 2022.
(60) Provisional application No. 63/482,868, filed on Feb. 2, 2023.

(51) Int. Cl. A61B 5/024; A61B 5/00; A61B 5/021; A61B 5/0245; A61B 5/352

(52) U.S. Cl. CPC A61B 5/02438; A61B 5/02116; A61B 5/02427; A61B 5/0245; A61B 5/352; A61B 5/6826; A61B 5/6843; A61B 5/6898; A61B 5/7405; A61B 5/743; A61B 2562/0219; A61B 2562/0247

(57) ABSTRACT

Device, method, and system for cuffless blood pressure (BP) measurement are provided. The device, method, and system include the use of an oscillometric finger pressing method to determine blood pressure using smartphones or other readily available computing devices. Further devices, methods, and systems are provided to improve BP computation via modeling and/or the use of additional measurements of ECG and DC PPG.

### CROSS-REFERENCE TO RELATED APPLICATION

This application is a continuation of International Application No. PCT/US23/27390, filed Jul. 11, 2023, which claims priority to U.S. Provisional Patent Application Ser. No. 63/388,021, filed Jul. 11, 2022, and U.S. Provisional Patent Application Ser. No. 63/482,868, filed Feb. 2, 2023, all of which are hereby incorporated by reference herein in their entireties.

### STATEMENT REGARDING FEDERALLY SPONSORED RESEARCH OR DEVELOPMENT

This invention was made with government support under grant no. HL146470 awarded by the National Institutes of Health. The government has certain rights in the invention.

### FIELD

The presently disclosed subject matter relates to devices, systems, and methods for measuring blood pressure. The disclosed device and systems can perform the cuffless blood pressure measurement by using an oscillometric finger pressing or hand raising technique to determine blood pressure.

### BACKGROUND

High blood pressure (BP) afflicts about one in three adults worldwide. While the incidence increases with age, many people develop hypertension early in adulthood (e.g., more than one in five US adults under 40 years old are hypertensive). The condition is usually asymptomatic, but the risk for stroke and heart disease increases monotonically with BP for a given age. Lifestyle changes and many inexpensive, once-daily medications can lower BP and cardiovascular risk. Yet, only about three in seven people with hypertension are aware of their condition, and just one of these seven has their BP under control. Epidemiological data on hypertension in low-resource settings are more alarming. As a result, hypertension has emerged as the leading cause of disability-adjusted life years lost.

Auscultatory and oscillometric BP measurement devices have been instrumental in managing hypertension. At the same time, these devices can bear responsibility for the abysmal hypertension awareness and control rates due to their reliance on an inflatable cuff. Cuff-based devices are not readily available, especially in low-resource settings. Hence, most people do not regularly check their BP. Regular measurements during daily life are needed to circumvent white coats and masked effects in the clinic in which patients present with higher or lower BP than usual and to average out the large variations in BP that occur over time due to stress, physical activity, and other factors. If BP could be measured more conveniently, then many people would become aware of their condition or motivated to take their medications.

Hence, cuffless BP monitoring devices are being widely pursued. However, the devices under investigation generally suffer from the debilitating limitation of requiring calibrations with cuff devices in order to output a measurement in units of mmHg.

### SUMMARY OF THE DISCLOSED SUBJECT MATTER

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

To achieve these and other advantages and in accordance with the purpose of the disclosed subject matter, as embodied and broadly described, the disclosed subject matter includes a device, method, and system for blood pressure monitoring.

The disclosed subject matter provides a method for determining diastolic blood pressure of a user using a device with a photoplethysmography (PPG)-force sensor unit. The method can include providing visual or audio instructions with the device, measuring PPG oscillations of the finger and the finger pressures with the PPG-force sensor unit, computing a width of each of the PPG oscillations as a function of the finger pressure, computing diastolic blood pressure using the PPG oscillation width versus finger pressure function, and outputting the diastolic blood pressure on a graphical user interface of the device or sending the diastolic blood pressure to a database repository. In non-limiting embodiments, the instructions can instruct the user to position a finger on the PPG-force sensor unit and to press the finger on the PPG-force sensor unit at varying finger pressures.

In certain embodiments, the method for determining diastolic blood pressure can further include detecting a bend in the PPG oscillation width to the finger pressure function. In non-limiting embodiments, the bend in the PPG oscillation width to the finger pressure function can be detected by fitting at least two curves to the function and using an intersection of the curves.

In certain embodiments, the width of each PPG oscillation can be computed as an area-to-height ratio of the oscillation.

In certain embodiments, the method for determining diastolic blood pressure can further include measuring an electrocardiogram (ECG) with additional electrodes incorporated into the device and computing the width of each PPG oscillation as a pulse arrival time for each of the oscillations detected as a time delay between an R-wave of the ECG and a PPG foot.

In certain embodiments, the method for determining diastolic blood pressure can further include determining systolic blood pressure by using a value of the pulse arrival time at the bend of the pulse arrival time to the finger pressure function.

In certain embodiments, the diastolic blood pressure can be computed using additional features extracted from the PPG oscillations and the finger pressure. In non-limiting embodiments, the additional features can include a finger pressure at a maximum slope of the PPG oscillation amplitude to finger pressure function.

The disclosed subject matter provides a method for determining systolic blood pressure of a user using a device with a photoplethysmography (PPG)-force sensor unit and electrocardiogram (ECG) electrodes. The method can include providing visual or audio instructions with the device, measuring a total PPG including a direct current (DC) component, PPG oscillations, and the applied finger pressure with the PPG-force sensor unit, measuring ECG simultaneously with the electrodes, computing an average of each PPG beat over the R-wave to R-wave interval of the ECG as a function of the applied finger pressure, and computing the systolic blood pressure using the PPG average to the finger pressure function. In non-limiting embodiments, the instructions can instruct the user to position a finger on the PPG-force sensor unit and to press the finger on the PPG-force sensor unit at varying finger pressures.

In certain embodiments, the method for determining systolic blood pressure can further include computing systolic blood pressure comprises detecting a bend in the PPG average to the finger pressure function. In non-limiting embodiments, the bend in the PPG average to the finger pressure function can be detected by fitting at least two curves to the function and using an intersection of the curves. In non-limiting embodiments, the systolic blood pressure can be computed using additional features extracted from the total PPG, finger pressure, and ECG. In non-limiting embodiments, the additional features can include a finger pressure at a minimum slope of the PPG oscillation amplitude to finger pressure function. In non-limiting embodiments, the additional features can include a value of the pulse arrival time at the bend of the pulse arrival time to the finger pressure function.

The disclosed subject matter provides a method for determining the pulse pressure of a user using a device. The method can include providing visual or audio instructions with the device to the user to position a finger on a camera and adjacent screen of the device to measure a total PPG from the finger via the camera of the device and the finger contact parameters via a touch screen sensor of the screen, providing visual or audio instruction to the user to apply a finger pressure on the camera and screen based on the measurements, providing visual or audio instructions to the user with the device to lower or raise a hand with respect to a heart level of the user while maintaining the finger pressure, measuring a hydrostatic blood pressure change (pgh) in the finger by using an accelerometer measurement of the device and an arm length, and computing pulse pressure from the PPG and pgh measurements.

In certain embodiments, the method for determining pulse pressure can further include providing an instruction with the device for a one-time initialization to determine an optimal placement guide for the finger on the camera and the screen.

In certain embodiments, the finger contact parameters can include a touch centroid. In non-limiting embodiments, the PPG oscillations can be used as a guide to determine the amount of finger pressure on the screen and camera to be maintained during hand raising or lowering. In non-limiting embodiments, the PPG oscillations and the finger contact parameters can be used as a guide to determine the amount of finger pressure on the screen and camera to be maintained during hand raising or lowering. In non-limiting embodiments, the finger contact parameters can be used as the guide to maintain the finger pressure on the screen and the camera during the hand raising or lowering. In non-limiting embodiments, a timer can be used as a further guide to indicate the amount of time to take for hand raising or lowering.

In certain embodiments, the pulse pressure can be computed using at least one of pgh at minimum and maximum slopes of the PPG oscillation amplitude to pgh function or a width of the PPG oscillation amplitude as a function of pgh.

In certain embodiments, the method for determining pulse pressure can further include measuring diastolic blood pressure by measuring a maximal finger contact parameter via firm finger pressing by the user and comparing the finger contact parameter with a maximal area to determine if diastolic blood pressure is low.

The disclosed subject matter provides a method for determining blood pressure using a device with a photoplethysmography (PPG)-force sensor unit and ECG electrodes. The method can include receiving a pressure measurement from a touch of a finger on the PPG-force sensor unit, receiving ECG from the electrodes, computing diastolic blood pressure with a processor of the device using a width of each alternative current (AC) blood volume oscillation versus finger pressure function, computing by the processor systolic blood pressure using an average of each direct current (DC) blood volume beat over the RR interval of the ECG versus finger pressure function, and outputting the blood pressure on a graphical user interface of the device or sending the blood pressure to a database repository.

The disclosed subject matter provides a system for determining blood pressure of a subject. The system can include sensors configured to measure finger pressure, a finger photoplethysmography (PPG) oscillations, finger PPG DC component, and ECG, a display configured to provide visual or audio instructions to guide the subject to position a finger at a predetermined location, and a processor. In non-limiting embodiments, the processor can be configured to compute diastolic blood pressure and systolic blood pressure and display the computed diastolic blood pressure and the systolic blood pressure on display. In non-limiting embodiments, the diastolic blood pressure can be calculated using a PPG oscillation width versus finger pressure function. In non-limiting embodiments, the systolic blood pressure can be calculated using a PPG average.

It is to be understood that both the foregoing general description and the following detailed description and drawings are examples and are provided for the purpose of illustration and not intended to limit the scope of the disclosed subject matter in any manner.

The accompanying drawings, which are incorporated in and constitute part of this specification, are included to illustrate and provide a further understanding of the devices of the disclosed subject matter. Together with the description, the drawings serve to explain the principles of the disclosed subject matter.

### BRIEF DESCRIPTION OF THE DRAWINGS

The subject matter of the application will be more readily understood from the following detailed description when read in conjunction with the accompanying drawings in which:

FIG. 1 is a diagram illustrating the oscillometric finger-pressing method for cuffless and calibration-free monitoring of blood pressure (BP) via readily available devices, such as a smartphone.

FIG. 2A is a diagram illustrating the smartphone-based device with a custom PPG-force sensor unit to implement the oscillometric finger pressure method. FIG. 2B is a diagram illustrating comparisons of the cuffless device to cuff devices.

FIGS. 3A-3C are diagrams illustrating the iPhone X app to implement the oscillometric finger pressing method via PPG and force sensors already available in the high-end phone.

FIG. 4 is a diagram illustrating a model of oscillometry and the oscillogram in particular. The oscillogram is nominally a function relating blood volume oscillation amplitude to the external pressure of an artery.

FIGS. 5A-5D are diagrams illustrating the differences in the model parameters for the finger and traditional arm site.

FIG. 6 is a diagram illustrating the well-known maximum amplitude and derivative algorithms for computing BP values from the oscillogram as well as the model predictions on how these algorithms perform.

FIG. 7 is a diagram illustrating model-predicted oscillations with increasing DP.

FIG. 8 is a diagram illustrating how the model can be modified to predict DC PPG and AC PPG oscillations with increasing external pressure.

FIG. 9 is a diagram illustrating a prototype device to make additional measurements of ECG and DC PPG (as opposed to conventional PPG oscillations alone).

FIG. 10 is a diagram illustrating methods to measure finger DP and SP from PPG oscillations, finger pressure, and ECG during actuation, such as finger pressing.

FIG. 11 is a diagram illustrating that P_{PAT}, P_{AHR}, and Pₘₐₓₛₗₒₚₑ (defined in FIG. 10) correlate well with arm cuff DP (N=34).

FIG. 12 is a diagram illustrating that P_{PAT} and P_{AHR} are indicators of DP in the finger.

FIG. 13 is a diagram illustrating that P_{PAT} is more robust than P_{AHR} during subject motion (N=4), as ECG is a robust sensing method.

FIG. 14 is a diagram illustrating that P_{PAT}, P_{AHR}, and Pₘₐₓₛₗₒₚₑ can be combined via simple averaging to further improve the accuracy of DP computation (N=34).

FIG. 15 is a diagram illustrating that Pₘᵢₙₛₗₒₚₑ combined with PAT at a fixed finger pressure (e.g., at P_{PAT}) correlates with arm cuff SP better than Pₘᵢₙₛₗₒₚₑ alone (N=34). The results in each plot of the figure were obtained via leave-one-out cross-validation.

FIG. 16 is a diagram illustrating a method to measure finger SP from the total PPG and finger pressure during actuation, such as finger pressing.

FIG. 17 is a diagram illustrating that P_{DCpeak} correlates well with arm cuff SP (N=14).

FIG. 18 is a diagram illustrating the total PPG versus finger pressure during actuation, such as finger pressing with the hands raised, at heart level, and lowered (N=2).

FIG. 19 is a diagram illustrating a method to convert finger BP to arm BP.

FIG. 20 is a diagram illustrating an innovative app for measuring pulse pressure (PP=SP−DP) using only a standard smartphone (without 3D Touch capabilities).

FIG. 21 is a diagram illustrating a Samsung Galaxy S21 app that measures PPG (R, G, and B AC+DC channels), accelerometry (X, Y, and Z axes) and finger contact area (major and minor radius, x- and y-center position).

FIG. 22 is a diagram illustrating exemplary measurements of PP using the guidance system described in FIG. 20 (N=3).

FIG. 23 is a diagram illustrating that the base of the fingernail positioned on top of the front camera is a suitable default position for obtaining large amplitude PPG oscillations.

FIG. 24 is a diagram illustrating ideal sensors for testing an oscillometric hand-raising method for measuring pulse pressure (PP).

FIG. 25 is a diagram illustrating a three-step procedure to compute PP.

FIG. 26 is a diagram illustrating the cuffless PP (width) versus arm cuff PP (N=22). The correlation coefficient (r) was 0.75. The mean and standard deviation of the PP errors were −2.9 and 7.4 mmHg.

FIG. 27 are graphs illustrating example models for improving oscillometric BP computation.

FIG. 28 is a diagram illustrating PPG oscillation during increasing pressure.

FIG. 29 are graphs illustrating example model predictions for collapsible finger arteries.

FIG. 30 are graphs illustrating the expansion of the disclosed model to AC+DC PPG and prediction.

FIG. 31 are graphs illustrating the model-based DP and SP computation algorithms.

FIG. 32 shows images illustrating an example benchtop system for assessing the disclosed device and models.

FIG. 33 are graphs illustrating the accuracy of the disclosed models and algorithms.

FIG. 34 are graphs illustrating the DC PPG results and the correlation between systolic and diastolic references.

FIG. 35 are graphs illustrating the DC PPG results using leave-one-out (LOO) regression.

FIG. 36 are graphs illustrating the correlation between the Minslope prediction and the vologram bend measurement.

FIG. 37 are graphs illustrating an example vologram bend measurement.

### DETAILED DESCRIPTION

Reference will now be made in detail to embodiments of the disclosed subject matter, an example of which is illustrated in the accompanying drawings. The disclosed subject matter will be described in conjunction with a detailed description of the system.

The terms used in this specification generally have their ordinary meanings in the art, within the context of the disclosed subject matter, and in the specific context where each term is used. Certain terms are discussed below or elsewhere in the specification to provide additional guidance to the practitioner in describing the compositions and methods of the disclosed subject matter.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing,” and “comprising” are interchangeable, and one of the skills in the art is cognizant that these terms are open-ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

A “user” or “subject” herein is a vertebrate, such as a human or non-human animal, for example, a mammal. Mammals include, but are not limited to, humans, primates, farm animals, sport animals, rodents, and pets.

The disclosed subject matter provides systems and techniques for determining the blood pressure of a subject. For example, the disclosed subject matter provides techniques for determining diastolic blood pressure, systolic blood pressure of a user, and/or pulse pressure of a user using a device with a photoplethysmography (PPG)-force sensor unit.

In certain embodiments, the method for determining the diastolic blood pressure of a user can include providing visual or audio instructions that can instruct the user to position a finger on the PPG-force sensor unit and to press the finger on the PPG-force sensor unit at varying finger pressures. The PPG-force sensor unit can be configured to measure the PPG of the user and the applied finger pressure. The PPG-force sensor unit can include a camera PPG sensor, a finger PPG sensor, a force sensor, or a combination thereof. The PPG-force sensor unit can be configured to detect PPG oscillations and the applied finger pressure from the subject's finger on the PPG sensor unit. In non-limiting embodiments, the PPG sensor can obtain the PPG oscillations without using a force sensor.

In certain embodiments, the method for determining diastolic blood pressure can include measuring PPG oscillations of the finger and the applied finger pressures with the PPG-force sensor unit. In non-limiting embodiments, the PPG oscillation can be a variable amplitude PPG oscillation. For example, since PPG oscillations can be proportional to blood volume oscillations in the artery and the artery volume-pressure relationship is nonlinear (sigmoidal), when sweeping the applied finger pressure, the PPG oscillation amplitude can increase to a maximum and then decrease to zero when the artery is occluded. In non-limiting embodiments, the method for determining diastolic blood pressure can include computing a width of each of the PPG oscillations as a function of the applied finger pressure, computing diastolic blood pressure using the PPG oscillation width versus finger pressure function, and outputting the diastolic blood pressure on a graphical user interface of the device or sending the diastolic blood pressure to a database repository. For example, the variable-amplitude PPG oscillations can be obtained from the PPG waveform as the user presses their finger on the sensors to vary the external pressure of the underlying artery. In non-limiting embodiments, the finger PPG waveform can include alternating current (AC) and direct current (DC) components during increasing external finger pressure. In non-limiting embodiments, the AC component can reflect the pulsation of blood, and the DC component can reflect the non-pulsatile component of the PPG signal. In non-limiting embodiments, the DC and/or AC components of the PPG waveform can be used to determine how much finger pressure the user needs to apply on the PPG sensor (e.g., camera or finger PPG sensor). For example, the user can first press hard on the PPG sensor to determine the highest DC value based on the user input. The device can show a graph for recording the DC value versus time, where the y-axis range can be set by the identified highest DC level. The processor can determine the DC level at which the AC oscillation amplitude is greatest, which can correspond to mean BP and show a constant target line to guide the user in attaining this level of substantial transmural pressure (external-internal pressure of the underlying artery) by pressing their finger or raising their hands.

In certain embodiments, the method for determining diastolic blood pressure can further include detecting a bend (i.e., a sudden change in the slope of the function) in the PPG oscillation width to the finger pressure function. For example, the bend in the PPG oscillation width to the finger pressure function can be detected by fitting at least two curves to the function and using an intersection of the curves.

In certain embodiments, the width of each PPG oscillation can be computed as an area-to-height ratio of the oscillation. For example, the area can be defined as area between a PPG waveform beat trough to trough and a line between the two troughs. The height can be defined as the trough-to-peak amplitude of the PPG wavefrom beat. The area-to-height ratio can be the ratio between the two. In non-limiting embodiments, the method for determining diastolic blood pressure can include measuring an electrocardiogram (ECG) with additional electrodes incorporated into the device, and computing the width of each PPG oscillation as a pulse arrival time for each of the oscillations as a time delay between an R-wave of the ECG and a PPG foot. For example, pulse arrival time (PAT), which is the time delay between ECG R-wave and PPG foot, and area-to-height ratio (AHR) are both indicators of oscillation width. When PAT and AHR are plotted against finger pressure to yield “PAT and AHR oscillograms,” fiducial markers can be identified to denote finger diastolic pressure (DP). For example, two lines/curves can be fitted to each of these oscillograms, and the intersection (P_{PAT} Or P_{AHR}) gives the DP. In non-limiting embodiments, the method can further include determining systolic blood pressure using a value of the pulse arrival time at a bend of the pulse arrival time to the finger pressure function. Systolic can be computed with an inversely proportional relationship to the pulse arrival time and proportional to the height of the subject. The coefficients are found from population data.

In certain embodiments, diastolic blood pressure can be computed using at least one of the width algorithms and each of the additional features extracted from the PPG oscillations and finger pressure. The additional features can include a finger pressure at a maximum slope of the PPG oscillation amplitude to finger pressure function. For example, diastolic blood pressure can be computed using at least the pressure at the bend in the width vs pressure. For example, the pressure at the maximum (Pₘₐₓₛₗₒₚₑ) and the minimum slope (Pₘᵢₙₛₗₒₚₑ) of the standard oscillogram (“height oscillogram”) can indicate finger DP and SP, respectively.

The disclosed subject matter provides a method for determining the systolic blood pressure of a user using a device with a photoplethysmography (PPG)-force sensor unit and ECG electrodes. The method can include providing visual or audio instructions with the device, wherein the instructions instruct the user to position a finger on the PPG-force sensor unit and to press the finger on the PPG-force sensor unit at varying finger pressures and measure a total PPG including a direct current (DC) component, PPG oscillations, the applied finger pressure with the PPG-force sensor unit, and measuring ECG simultaneously with the electrodes.

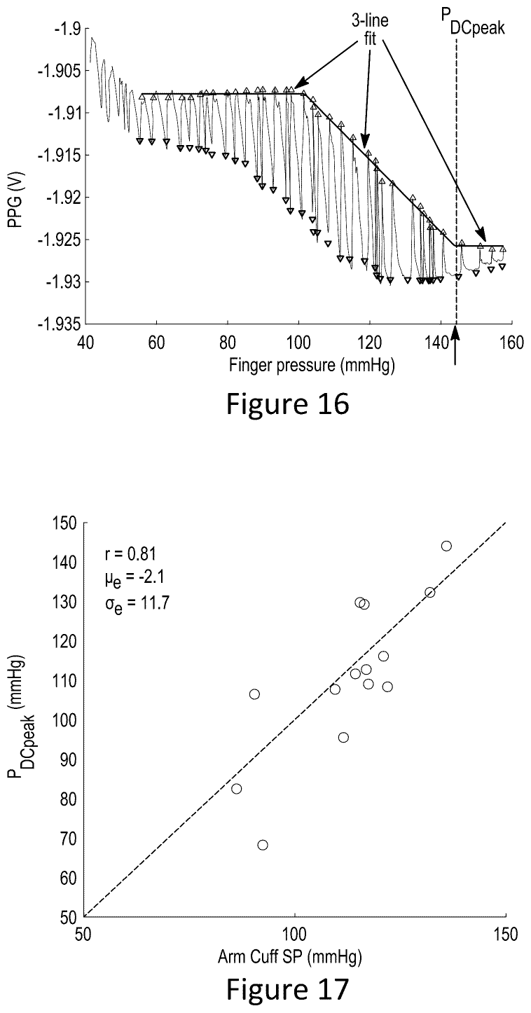

In certain embodiments, the method for determining the systolic blood pressure of a user can include computing an average of each PPG beat over the R-wave to R-wave interval of the ECG as a function of the applied finger pressure and computing the systolic blood pressure using the PPG average to the finger pressure function. In non-limiting embodiments, the PPG beat can reflect the pulsation of the PPG at every heart beat, and the average of the PPG beat can reflect the average value within that beat. For example, three lines can be fitted to the upper envelope of the plot relating total (DC+AC) PPG versus finger pressure, and the intersection of the second two lines (P_{DCpeak}) denotes finger SP (FIG. 16).

In certain embodiments, the method for determining systolic blood pressure can further include detecting a bend (i.e., a sudden change in the slope of the function) in the PPG average to the finger pressure function. For example, the bend in the PPG average to the finger pressure function can be detected by fitting at least two curves to the function and using an intersection of the curves.

In certain embodiments, the systolic blood pressure can be computed using additional features extracted from the total PPG, finger pressure, and ECG. In non-limiting embodiments, the additional features can include a finger pressure at a minimum slope of the PPG oscillation amplitude to finger pressure function. In non-limiting embodiments, the additional features can include a value of the pulse arrival time at the bend of the pulse arrival time to the finger pressure function. The pulse arrival time is defined as the time delay between the ECG R-wave to the PPG foot.

The disclosed subject matter provides a method for determining the pulse pressure of a user using a device. The method can include providing visual or audio instructions that can instruct the user to position the finger on a camera and adjacent screen of the device to measure a photoplethysmography (PPG) from the finger via the camera of the device and finger contact parameters via a touch screen sensor of the device, providing visual or audio instructions to the user to apply a finger pressure on the camera and screen based on the measurements, providing visual or audio instructions to the user with the device to lower or raise a hand with respect to a heart level of the user while maintaining the finger pressure guided with the finger contact parameters, and measuring a hydrostatic blood pressure change (pgh) in the finger by using an accelerometer measurement of the device and an arm length. In non-limiting embodiments, the finger contact parameters can include a touch centroid. The finger contact parameters are any parameters derive from the screen capacitive sensor array in a phone (e.g., touch centroid and touch area).

In certain embodiments, the method for determining pulse pressure can include computing pulse pressure from the PPG and the pgh measurements. For example, as a user lowers their hand with the arm straight, the internal blood pressure in the finger can increase due to the weight of the arm blood column (“hydrostatic effect”) by an amount equal to pgh, where p is the density of blood, g is the gravitational constant, and h is the vertical distance between the hand position and heart, and thus arterial transmural pressure can be varied. In non-limiting embodiments, the hydrostatic BP change (pgh) can be measured using an accelerometer and the length of the arm, since the angle of the arm according to the gravity vector can be computed from the accelerometer and using trigonometry with arm length, h can be computed. Alternatively, the hydrostatic BP change can be estimated without using an accelerometer/gyroscope or any other sensor. While maintaining the constant finger pressure, the user lowers the phone to the floor and raises it upwards in intuitive and fixed increments (e.g., approximately 45 degrees for 3-5 seconds at a time guided by audio/visual cues). The hydrostatic BP change can then be estimated based on the known increments.

In certain embodiments, the PPG oscillations can be used as a guide to determine the amount of finger pressure on the screen and camera to be maintained during hand raising or lowering. In non-limiting embodiments, the PPG oscillations and finger contact parameters can be used as a guide to determine the amount of finger pressure on the screen and camera to be maintained during hand raising or lowering. In non-limiting embodiments, the finger contact parameters can be used as the guide to maintain the finger pressure on the screen and camera during the hand raising or lowering. In non-limiting embodiments, a timer can be used as a further guide to indicate the amount of time to take for hand raising or lowering.

In certain embodiments, the pulse pressure can be computed using at least one of pgh at the minimum and maximum slopes of the PPG oscillation amplitude to pgh function or a width of the PPG oscillation amplitude as a function of pgh.

In certain embodiments, the method for determining pulse pressure can include measuring diastolic blood pressure by measuring the maximal finger contact parameter via firm finger pressing by the user and comparing the finger contact parameter with the maximal finger contact parameter to determine if diastolic blood pressure is low. For example, if the ratio between the maximum contact area and the contact area at a fiducial marker of diastolic (any algorithms to detect diastolic) is low, then diastolic can be low. If the ratio between the maximum contact area and the contact area at a fiducial marker of diastolic (any algorithm to detect diastolic) is high, then diastolic can be high. These ratios can be developed with population data. In non-limiting embodiments, the systolic pressure can be estimated with any of the proposed algorithms to compute pulse pressure by adding it to diastolic pressure.

In certain embodiments, the method for determining pulse pressure can include providing instructions for a one-time initialization to determine an optimal placement guide for the finger on the camera and screen. For example, the disclosed device can perform a one-time initialization phase to measure PPG oscillations across different thumb positions and thereby identify the best thumb placement. During the initialization, a user can be guided to incrementally place more of the finger on the screen. The device can identify the finger positioning that provides a suitable area of screen contact without approaching force saturation. For example and without limitation, the disclosed device can provide a visual indicator that can guide the user to place the finger that can yield the largest area of screen contact without force saturation. This initialization can also create a profile of the thumb and its box dimensions for guiding the thumb placement thereafter.

In certain embodiments, the blood pressure change in the finger can be measured when the device is at different verticals with respect to the heart level of the user. In non-limiting embodiments, the touch sensor adjacent to the camera can be a screen of the device. In non-limiting embodiments, the distance of the device from the heart of the user can be an arm length.

The disclosed subject matter provides a method for determining blood pressure using a device with a photoplethysmography (PPG)-force sensor and ECG electrodes. The method can include receiving measurements from a touch of a finger on the PPG-force sensor unit, receiving ECG from the electrodes, computing diastolic blood pressure (BP) with a processor of the device using a width of each alternative current (AC) blood volume oscillation versus finger pressure function, computing by a processor systolic BP using an average of each direct current (DC) blood volume beat over the RR interval of the ECG versus finger pressure function, and outputting the BP on a graphical user interface of the device or sending the BP to a database repository.

The disclosed subject matter provides a system for determining the blood pressure of a subject. The system can perform the disclosed methods for measuring blood pressure, DP, SP, pulse pressure, or combinations thereof. An example system can include sensors configured to measure finger pressure, finger photoplethysmography (PPG) oscillations, a finger PPG DC component, and ECG, a display configured to provide visual or audio instructions to instruct the user/subject to position a finger, a thumb, a hand, or combinations thereof at a predetermined location, and a processor configured to perform the disclosed methods of measuring blood pressure, DP, SP, pulse pressure, or combinations thereof.

In certain embodiments, the processor can be configured to compute diastolic blood pressure and systolic blood pressure and display the calculated diastolic blood pressure and systolic blood pressure on display. In non-limiting embodiments, the diastolic blood pressure can be calculated using a PPG oscillation width versus finger pressure function, and the systolic blood pressure can be calculated using a PPG average.

### EXAMPLES

### Example 1

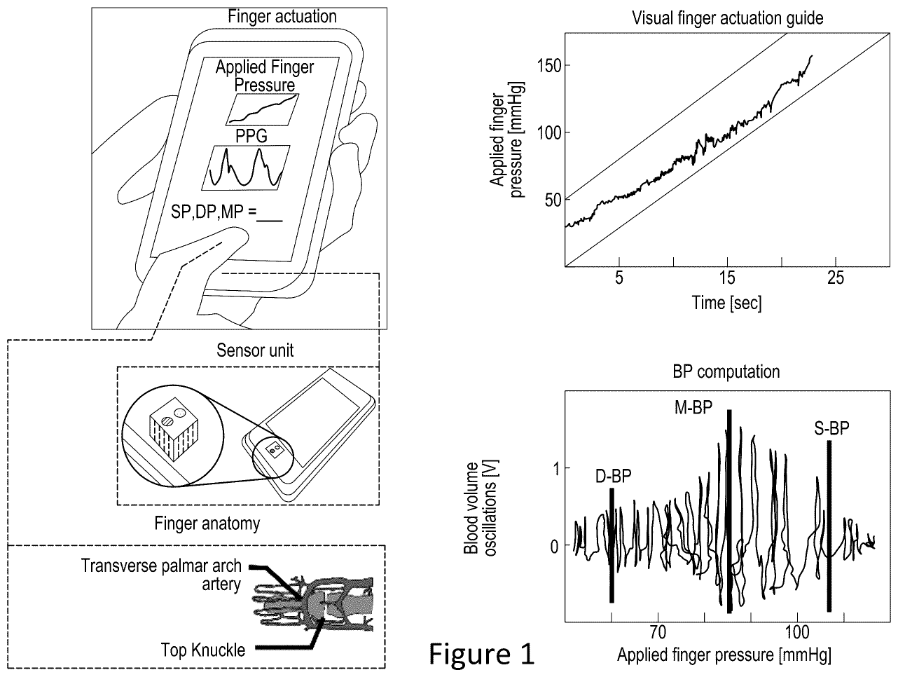

The disclosed subject matter builds upon oscillometric principles for cuffless and calibration-free BP monitoring via readily available handheld devices such as a smart wearable device, tablet, or smartphone, as shown in FIG. 1. Such devices can not only include processors that can compute and evaluate but also include photoplethysmography (PPG) and force transducers useful in BP monitoring. The user can serve as the actuator (instead of a cuff) by pressing their index fingertip against the device or phone (held at heart level) to steadily increase the external pressure of the underlying artery. The phone, embedded with photoplethysmography (PPG) and force transducers, serves as the sensor (rather than the cuff device) to measure the resulting variable-amplitude blood volume oscillations and applied finger pressure. The phone also provides visual feedback on the graphical user interface (GUI) or audio feedback via the phone speakers to guide the finger actuation and applies an algorithm to compute BP from the measurements comparable to the computation method of oscillometric cuff devices.

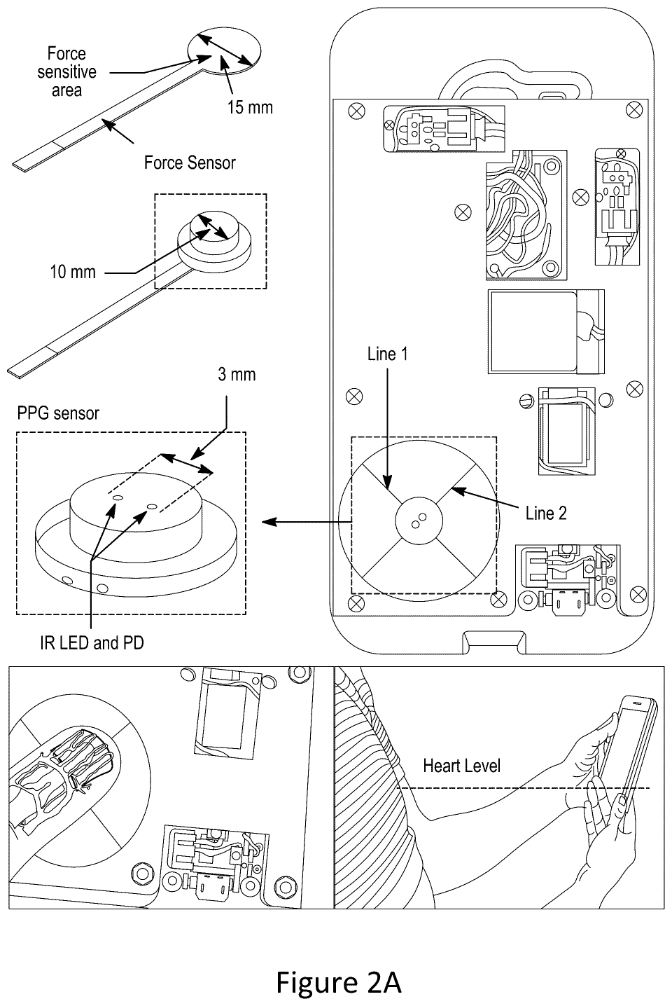

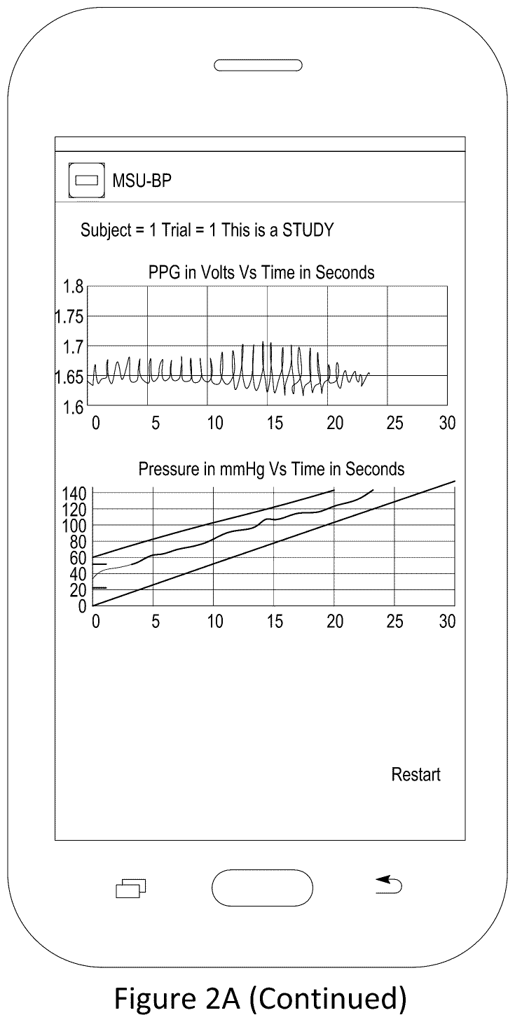

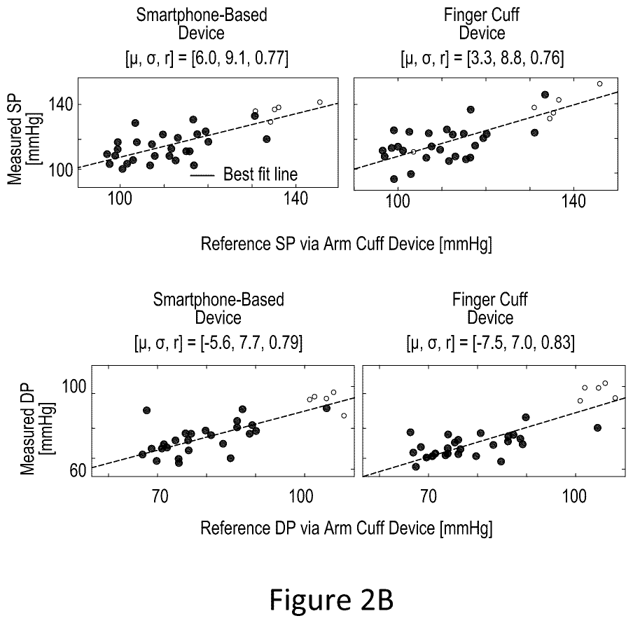

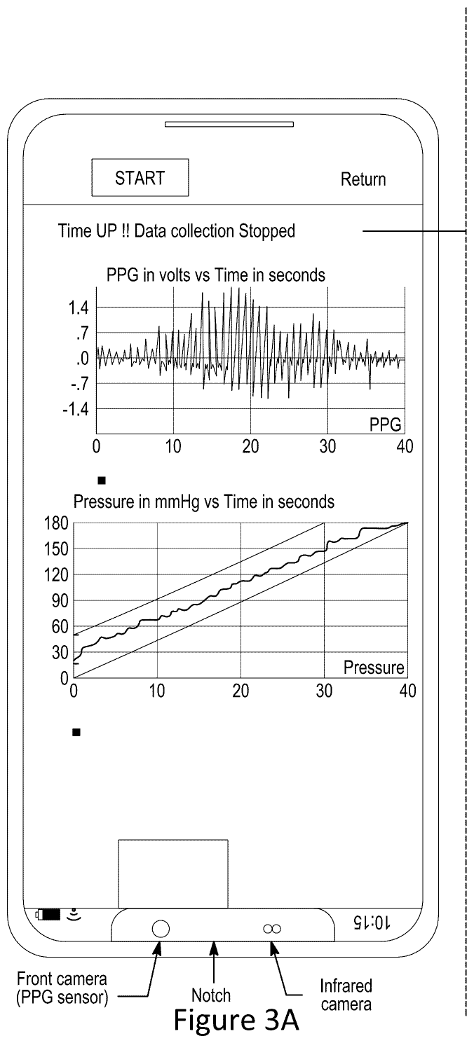

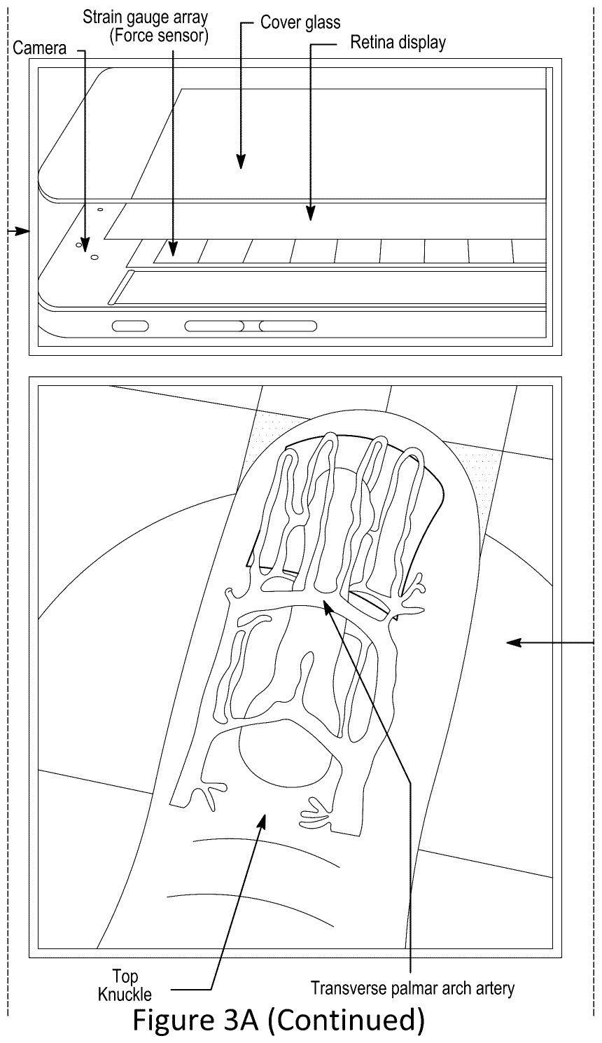

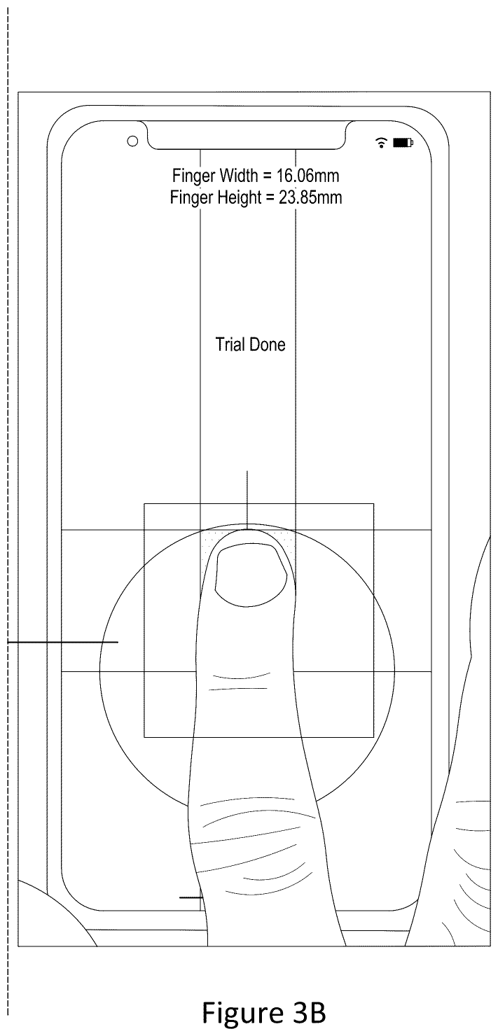

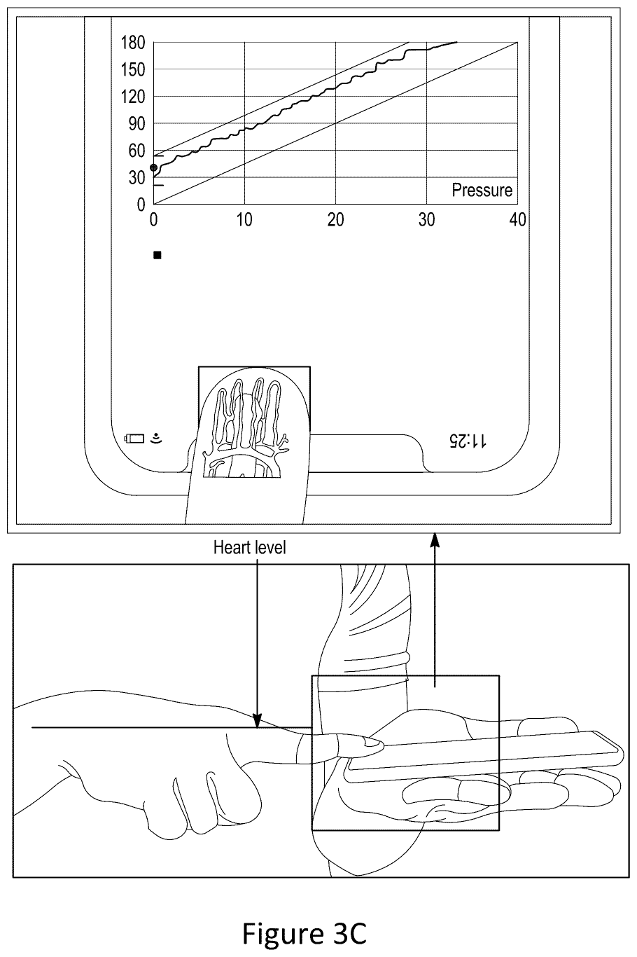

FIG. 2A shows a device comprised of a custom PPG-force sensor unit affixed to the back of a readily available device to implement the “oscillometric finger pressing method.” The device yields BP measurements with a level of accuracy comparable to an FDA-cleared finger cuff volume clamping device over the normotensive range, as shown in FIG. 2B. In non-limiting embodiments, the oscillometric finger pressing method can be implemented as a smartphone (like an iPhone) application (“app”), as shown in FIGS. 3A-3C, by leveraging the front camera as the PPG sensor and the sensitive strain gauge array (“3D Touch”) under the screen as the force sensor.

The disclosed subject matter provides a device, method, and system for improving the BP computation accuracy based on physiologic modeling. In certain embodiments, the method includes a related oscillometric hand raising/lowering method for cuffless and calibration-free monitoring of BP with standard devices like smartphones, where sensitive force sensing is usually not available. The disclosed method can overcome the deficiencies in certain devices where sensitive force sensing is unavailable. In other embodiments, the method includes guiding the user to properly perform the hand actuation.

### Oscillometric Modeling

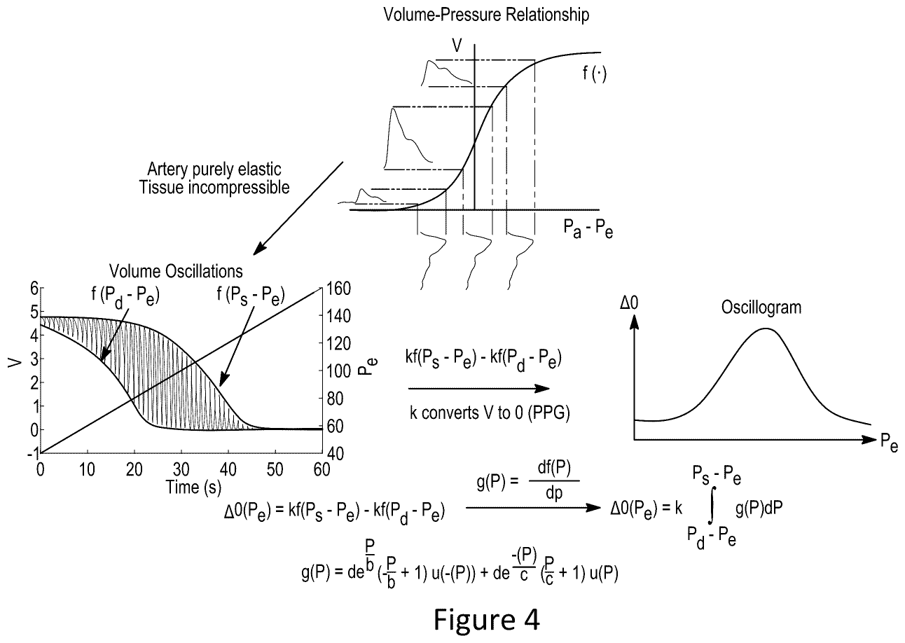

As shown in FIG. 4, the oscillometric model accounts for the sigmoidal blood volume-transmural pressure relationship of an artery, whereby transmural pressure includes the internal BP minus the external pressure of the artery. The model assumptions include a purely elastic arterial wall and a linear relationship between blood volume oscillations and the measured oscillations via PPG (or a cuff). In certain embodiments, the model can predict the oscillogram (i.e., oscillation amplitude versus external pressure function) for typical BP computation or the model can predict the oscillations.

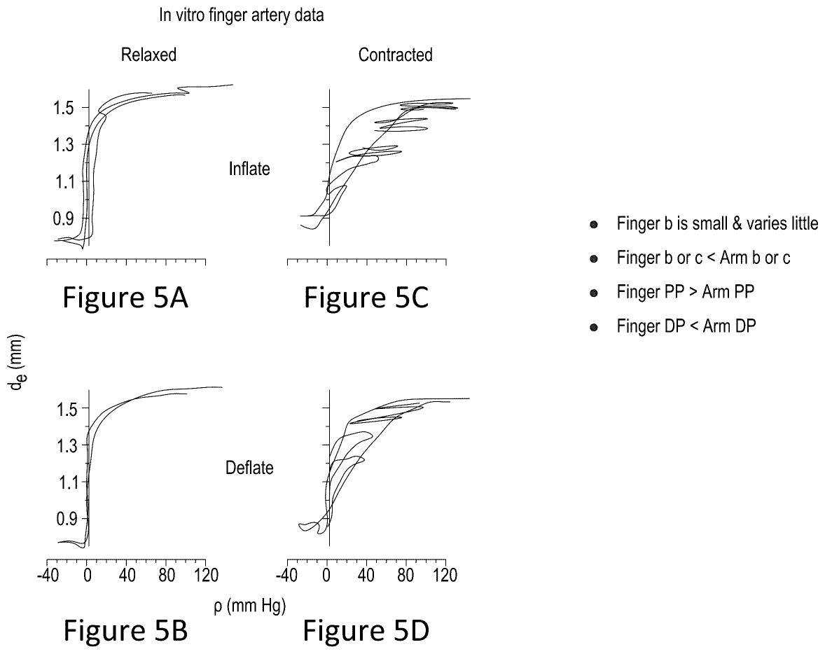

As shown in FIG. 5, the finger artery collapses when the external pressure is slightly above the internal BP (i.e., nearly “collapsible finger artery”). FIG. 5A indicates that the finger artery is collapsible in the sense that it is completely flattened when the external pressure is slightly above the internal BP. FIGS. 5B-5C refer to model parameters in FIG. 4. PP is pulse pressure, and DP is diastolic BP. Mathematically, this corresponds to a small b value for the finger in the model of FIG. 4. However, the external pressure can be significantly higher than the internal BP for larger arm artery collapse to occur. This difference is exploited to arrive at accurate finger oscillometric BP computation algorithms.

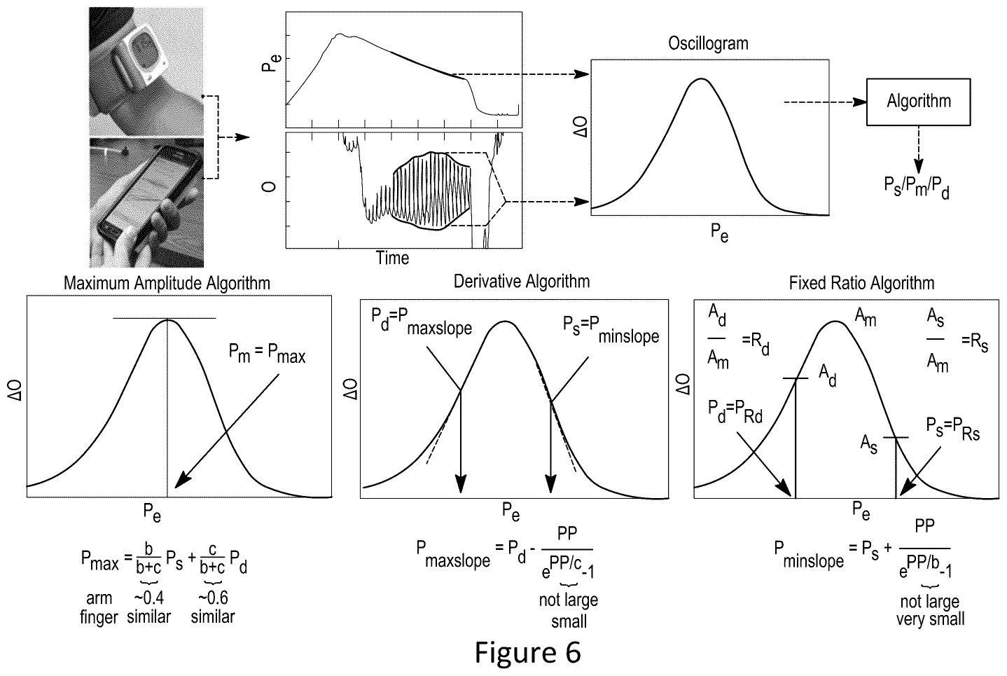

As shown in FIG. 6, the derivative algorithm is more accurate for the finger than the arm due to the model parameter differences. In particular, the external pressures at which the slopes of the oscillogram are maximal and minimal need to closely correspond to finger diastolic and systolic BP (DP and SP). However, the derivative operation can amplify measurement noise.

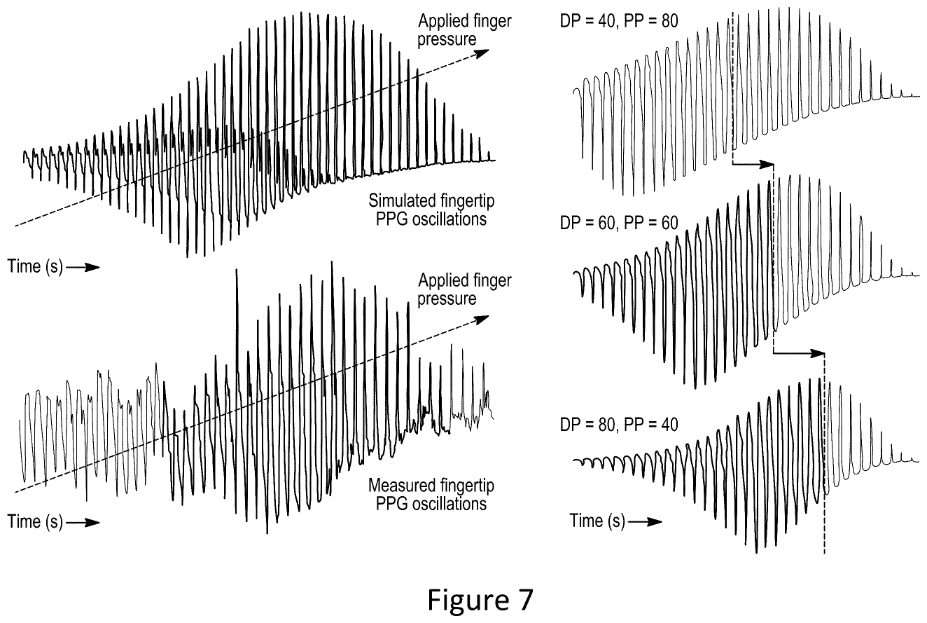

As shown in FIG. 7, due to finger collapsibility, the model predicts a narrowing of the width of each oscillation with increasing external pressure. But, as the DP increases, the narrowing occurs at higher external pressure. In certain embodiments, the narrowing begins at DP (or slightly above DP, as the finger artery is not perfectly collapsible). Similarly, the oscillations need to be abolished when the external pressure is slightly above SP.

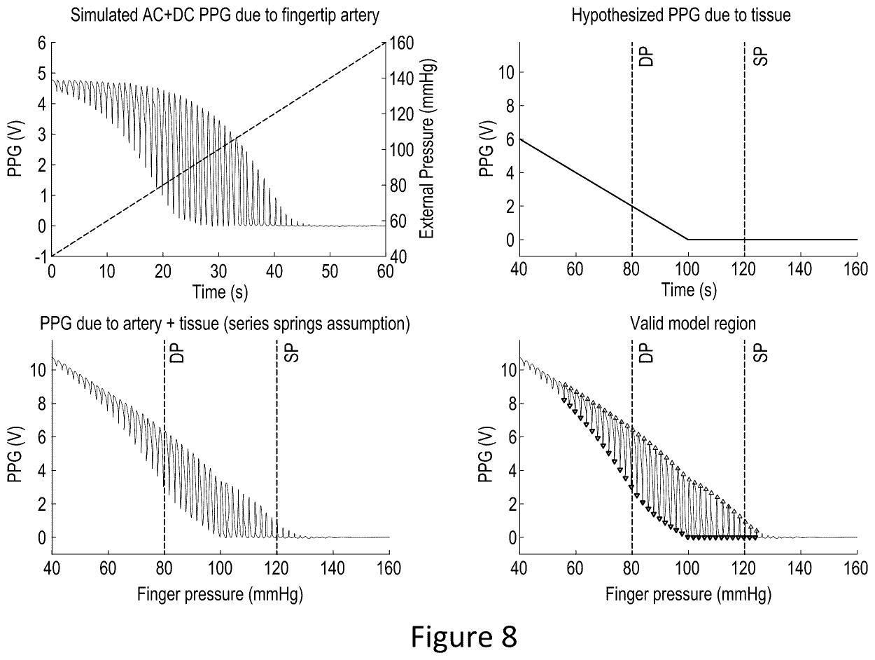

As shown in FIG. 8, the tissue surrounding the artery can be considered in predicting DC PPG plus the AC PPG oscillations with increasing external pressure. In particular, as the external pressure increases, the tissue compresses up to a certain point. As a result, DC PPG reduces and then saturates due to the tissue (upper right of the figure). The original artery model (upper left of the figure) can be added to this tissue model to predict the total PPG as external pressure increases (bottom).

### Improved Blood Pressure Computation

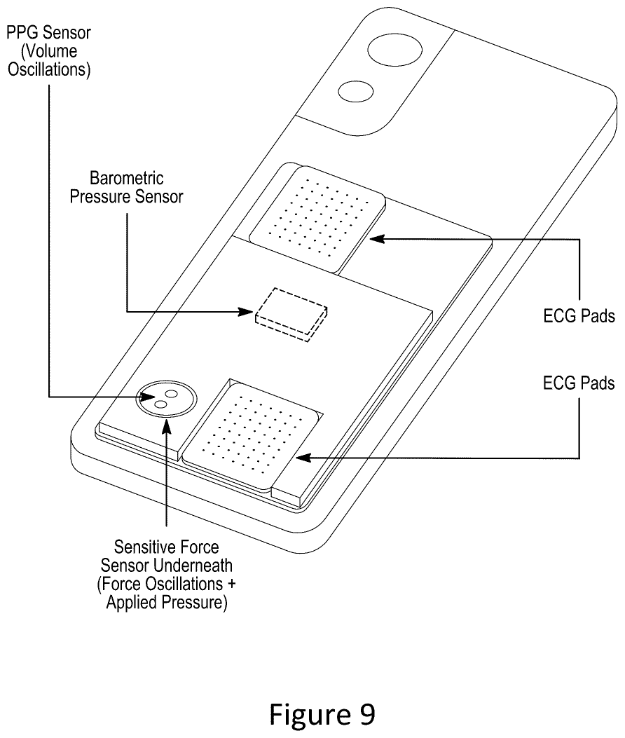

The disclosed subject matter provides improved methods to compute BP via modeling and/or the use of additional measurements of ECG and DC PPG (as opposed to conventional PPG oscillations alone). For purposes of example and not by limitation, a device, as shown in FIG. 9, can make the additional measurements. Additionally, an ECG can be beneficial for simultaneous BP and arrhythmia monitoring.

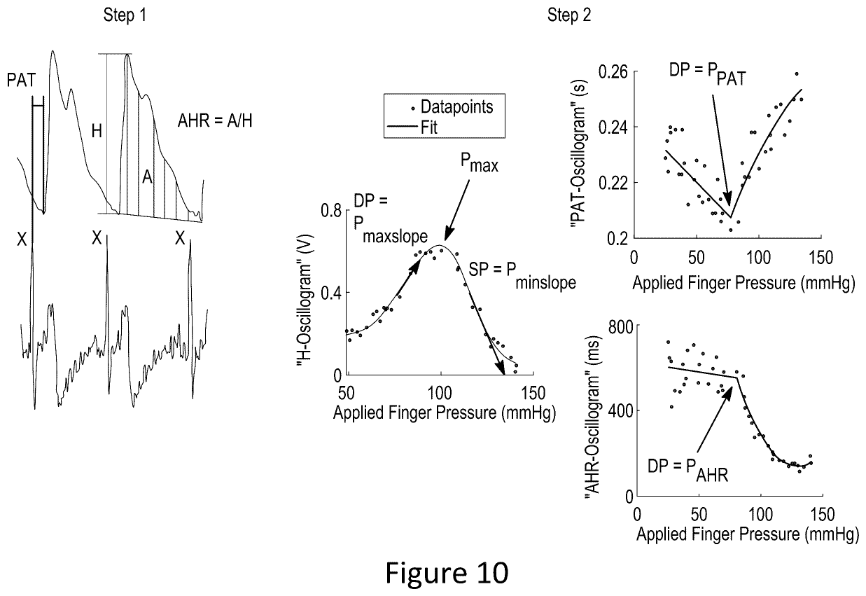

As shown in FIG. 10, pulse arrival time (PAT), which is the time delay between ECG R-wave and PPG foot, and area height ratio (AHR) are both indicators of oscillation width. When PAT and AHR are plotted against finger pressure to yield “PAT and AHR oscillograms,” clear fiducial markers are evident to denote finger DP. For example, two lines/curves can be fitted to each of these oscillograms, and the intersection (P_{PAT} Or P_{AHR}) gives the DP. Pₘₐₓₛₗₒₚₑ or Pₘᵢₙₛₗₒₚₑ of the standard oscillogram (“height oscillogram) respectively indicate finger DP and SP.

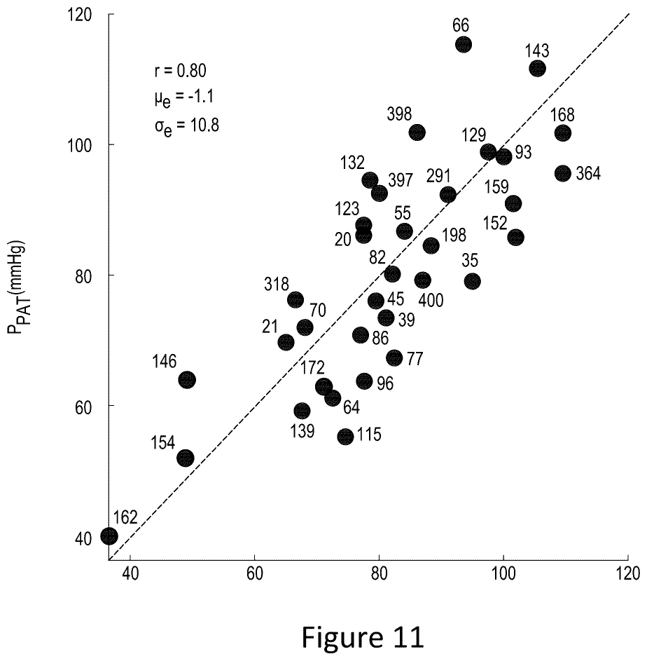

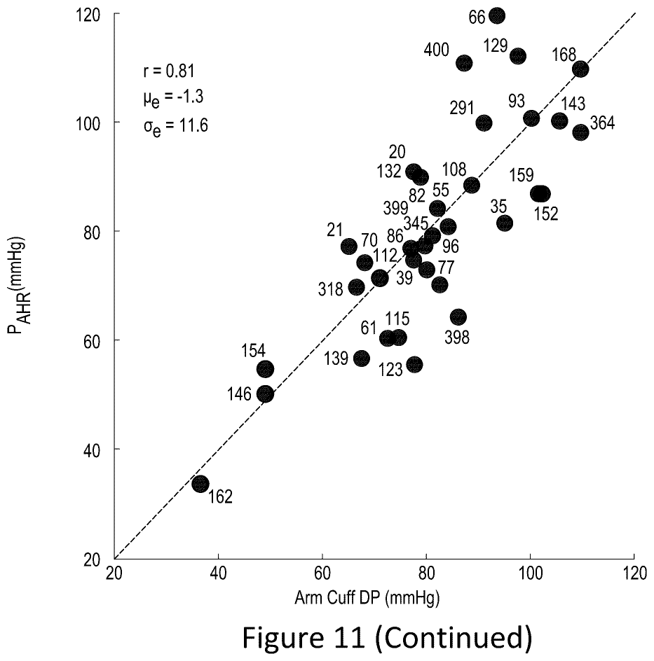

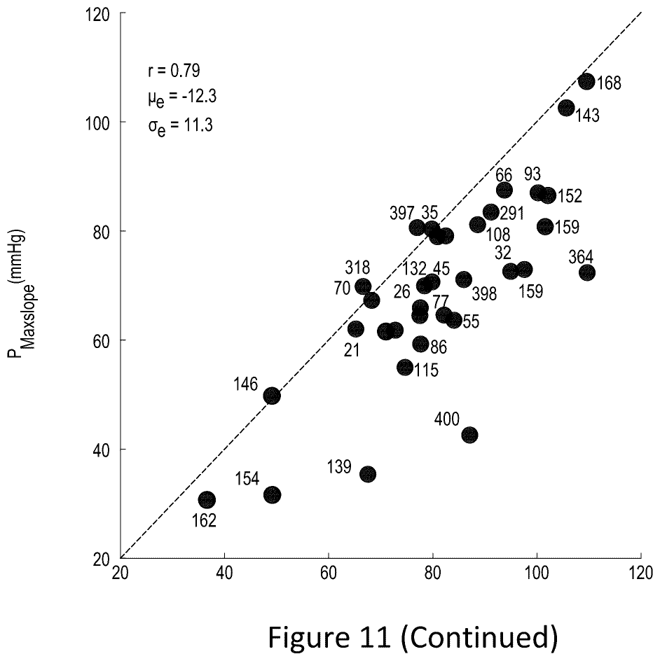

As shown in FIG. 11, P_{PAT}, P_{AHR}, and Pₘₐₓₛₗₒₚₑ correlate well with arm cuff DP (N=34). Arm cuff DP is systematically higher than finger DP, as shown in FIG. 5. P_{PAT} and P_{AHR} overestimate finger DP and their data points are near the identity line.

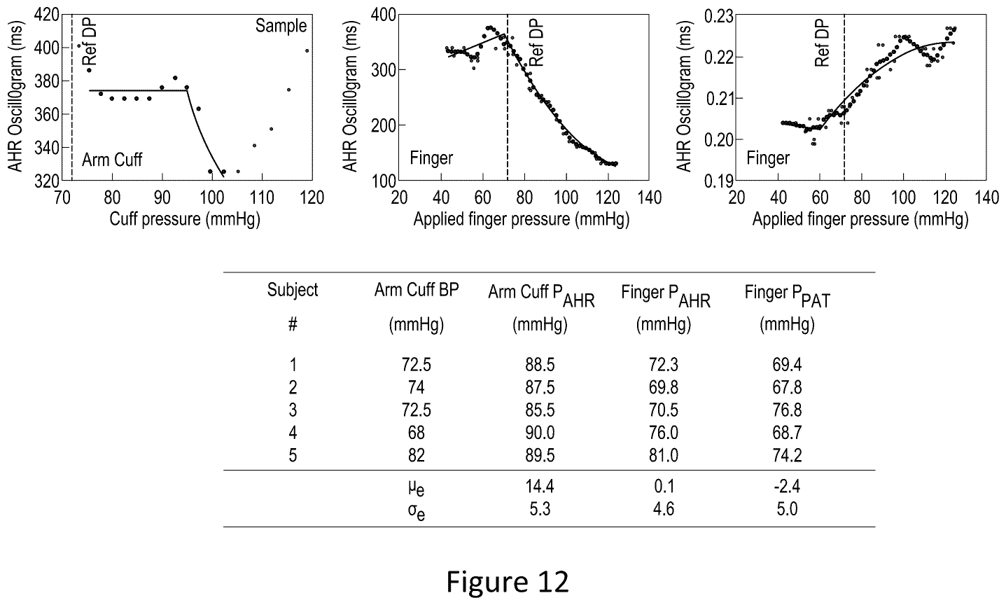

As shown in FIG. 12, P_{PAT} and P_{AHR} are indicators of DP in the finger but not the arm (N=5) because the larger arm artery is not collapsible.

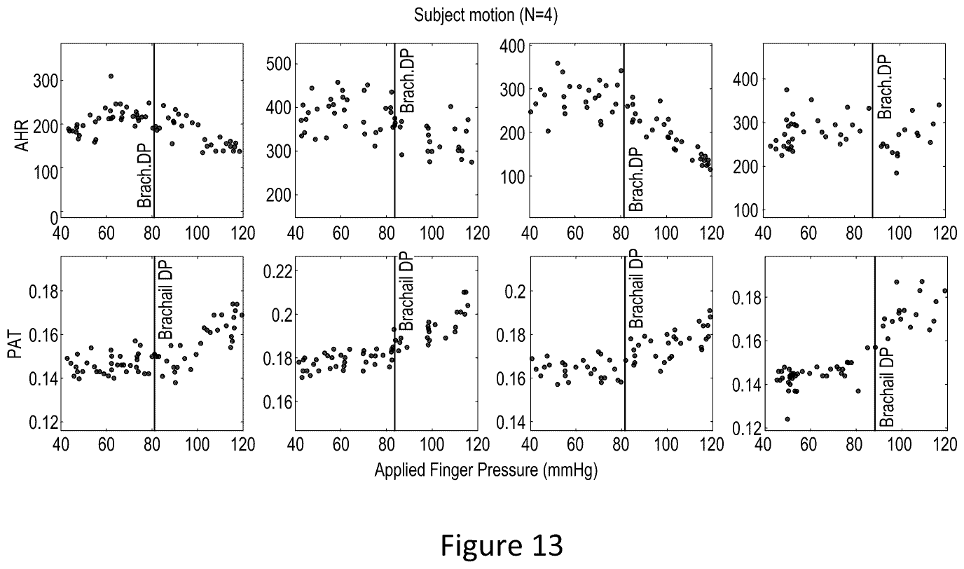

In one example, as shown in FIG. 13, P_{PAT} is depicted as being more robust than P_{AHR} during subject motion (N=4) because ECG is a particularly robust sensing method.

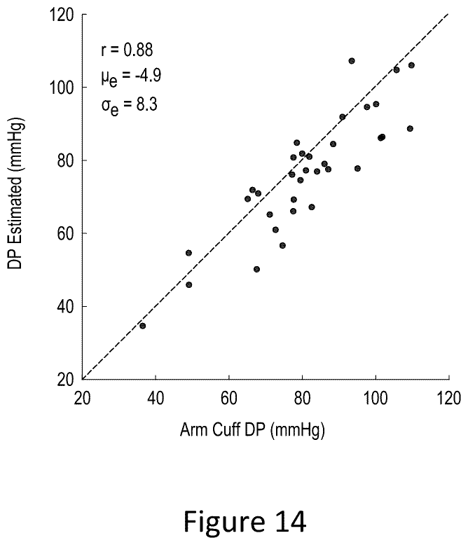

In certain embodiments, P_{PAT}, P_{AHR}, and Pₘₐₓₛₗₒₚₑ can be combined to further improve the accuracy of DP computation (N=34), as shown in FIG. 14. For example, the three indices can be averaged (“DP Estimated” as shown) or used as independent variables in a linear regression to predict DP. Alternatively, the two closest indices could be averaged.

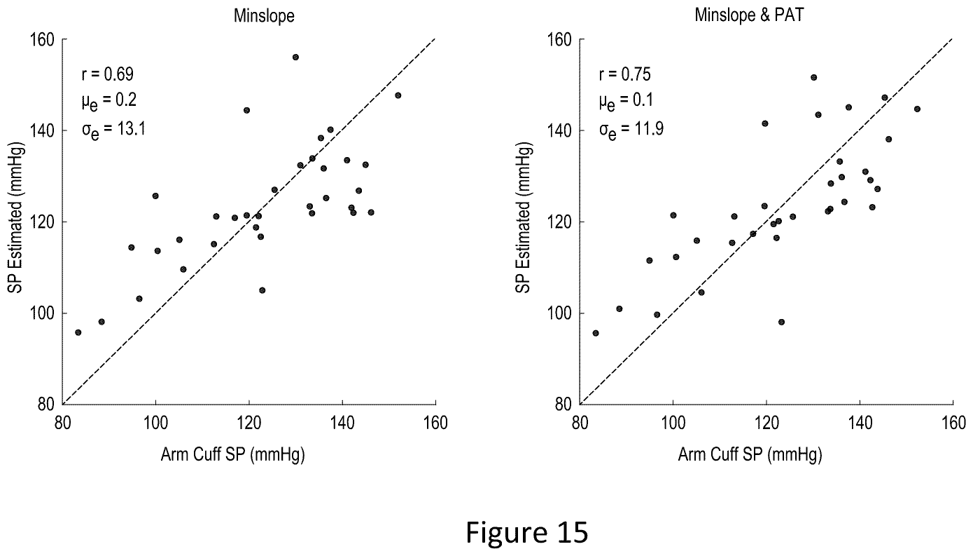

As shown in FIG. 15, Pₘᵢₙₛₗₒₚₑ combined with PAT at a fixed finger pressure (e.g., at P_{PAT} or P_{AHR}) correlates with arm cuff SP better than Pₘᵢₙₛₗₒₚₑ alone (N=34). PAT at fixed PPG sensor contact pressure is known to show some correlation with SP. In certain embodiments, Pₘᵢₙₛₗₒₚₑ and PAT at P_{PAT} can be combined in various ways including as independent variables in a linear regression (“SP Estimated” as shown in FIG. 15). Arm cuff SP is not as good a reference for finger SP as arm cuff DP is for finger DP, as indicated in FIG. 5.

In one example, as shown in FIG. 16, three lines are fitted to the upper envelope of the plot relating total (DC+AC) PPG versus finger pressure, and the intersection of the second two lines (P_{DCpeak}) denotes finger SP to measure finger SP from the total PPG and finger pressure. Three lines are fitted to the upper envelope of the plot relating total PPG versus finger pressure, and the intersection of the second two lines (P_{DCpeak}) denotes finger SP. The method assumes that this fiducial marker occurs after the tissue has been fully compressed (i.e., saturation in FIG. 8).

As shown in FIG. 17, P_{DCpeak} correlates well with arm cuff SP (N=14).

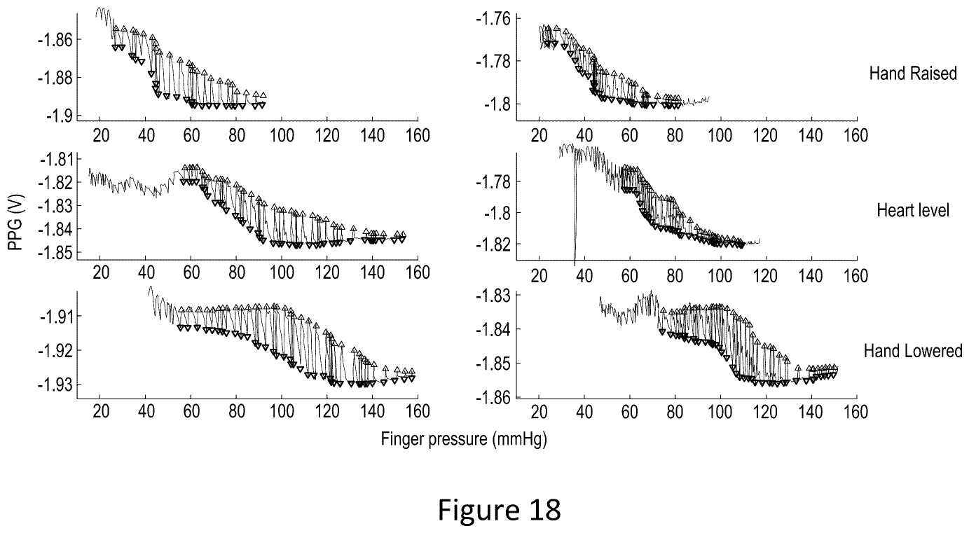

As shown in FIG. 18, the total PPG versus finger pressure plots change with the hands raised, at heart level, and lowered. Conventional plots at the heart level look similar to the model prediction of FIG. 8. The plots shift to the right due to the increased finger BP via the hydrostatic effect. The tissue can be fully compressed at some pressure (e.g., 80 mmHg) regardless of the vertical position of the hands. As a result, by artificially increasing the BP via lowered hands, the total PPG can be entirely due to the artery. The lower plots in the figure look like the model prediction due to the arterial component alone in FIG. 8 (upper left). For purposes of example and not by limitation, a method to estimate SP is to perform oscillometric finger pressing with the hands lowered fully, apply the method of FIG. 17 to detect finger SP, and correct the measurement to heart level by subtracting pgh, where p is the known blood density, g is gravity, and h is the length of the arm. In another embodiment, the method to estimate BP is to detect pulse pressure (PP=SP−DP) via powerful cross-correlation between the upper and lower envelopes. The front camera of the phone can be used to capture an image of the face during the finger pressing. This image can be used to verify whether the phone is being positioned correctly (i.e., with hands fully lowered) or not during the finger pressing by comparing it to a reference image.

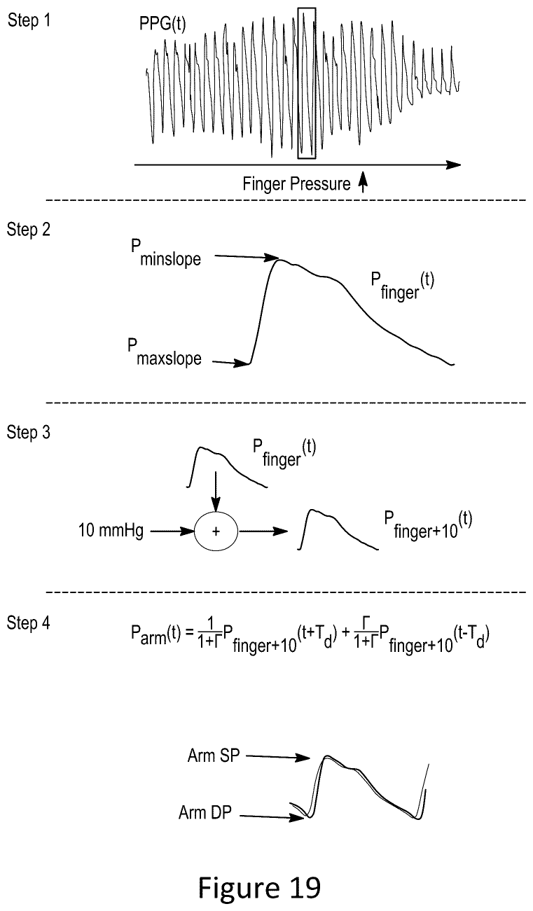

As shown in FIG. 19, arm BP can be computed from finger BP. The maximum oscillation can be where the sigmoidal blood volume-transmural pressure relationship is most linear and thus best denotes the finger BP oscillation shape (Step 1). Any of the above BP computations could be used instead of Pₘᵢₙₛₗₒₚₑ and Pₘₐₓₛₗₒₚₑ to calibrate the maximum oscillation (Step 2). The resistive pressure drop from the arm to the finger is first countered by adding a constant (e.g., 10 mmHg) to the finger BP (Step 3). A transfer function based on a physical tube-load model of arterial wave reflection is then applied to derive arm BP (Step 4). Γ and Γ_{d} are fixed values, for example, such as 0.3 and 72 msec. Alternatively, T_{d} is a function of a BP value, or the transfer function is based on a black-box model (e.g., autoregressive exogenous input model).

BP Apps for Readily Available Devices without Force Sensing

Given that every adult has a real risk for developing hypertension and that smartphones are available to billions of people including those in low-resource settings, it is desirable to convert standalone smartphones into BP monitors. However, many readily available devices, such as smartphones, do not include 3D Touch or similarly sensitive force sensors.

The idea to convert standard smartphones into absolute BP sensors is based on a previous method that uses arm rather than finger actuation. In traditional oscillometry, the cuff compresses the artery to vary its external pressure. During this process, the device also measures the cuff pressure, which indicates both the blood volume oscillations in the artery (AC cuff pressure) and the external pressure (DC cuff pressure). BP can be computed from the resulting oscillogram, which is the function relating the variable-amplitude blood volume oscillations to the applied pressure. The abscissa of the oscillogram can be viewed more generally as a change in the transmural pressure of the artery (internal BP minus external cuff pressure in this case). The previous method thus involves varying the internal rather than the external pressure of an artery to change the transmural pressure. As a user of a finger-worn ring device lowers their hand with the arm straight, the internal BP in the finger increases due to the weight of the arm blood column (“hydrostatic effect”) by an amount equal to pgh, where h is the vertical distance between the hand position and heart. In this way, arterial transmural pressure is varied without a cuff. The device includes a PPG sensor, force sensor, and accelerometer. The accelerometer and the length of the arm allow measurement of the hydrostatic BP change, pgh. The BP changes for typical arm lengths is about ±50 mmHg with respect to heart level. For a mean BP of 80 mmHg, the transmural pressure variation is about 30 to 130 mmHg. However, the oscillogram in both the positive and negative transmural pressure regimes is needed to compute BP accurately. Thus, the ring need to be worn tight enough to generate negative transmural pressures. The force sensor of the known area measures the ring contact pressure on the finger, which is subtracted from the hydrostatic BP change. BP can then be estimated from the PPG oscillations as a function of the transmural pressure change.

One problem is that the ring needs to be applied with a pressure equal to around mean BP, but BP is what is sought for measurement. Another problem with bringing the hand-raising actuation to a smartphone is eliminating the need for the force sensor. However, most readily available devices, such as smartphones, have PPG sensors in the form of a camera and a three-axis accelerometer/gyroscope combination.

To solve at least said noted problems, and as described in U.S. Provisional Application No. 63/135,430, the contents of which are incorporated by reference in its entirety, the measurement can be limited to PP. PP would be useful for detecting isolated systolic hypertension, which is a common form of hypertension that occurs with aging.

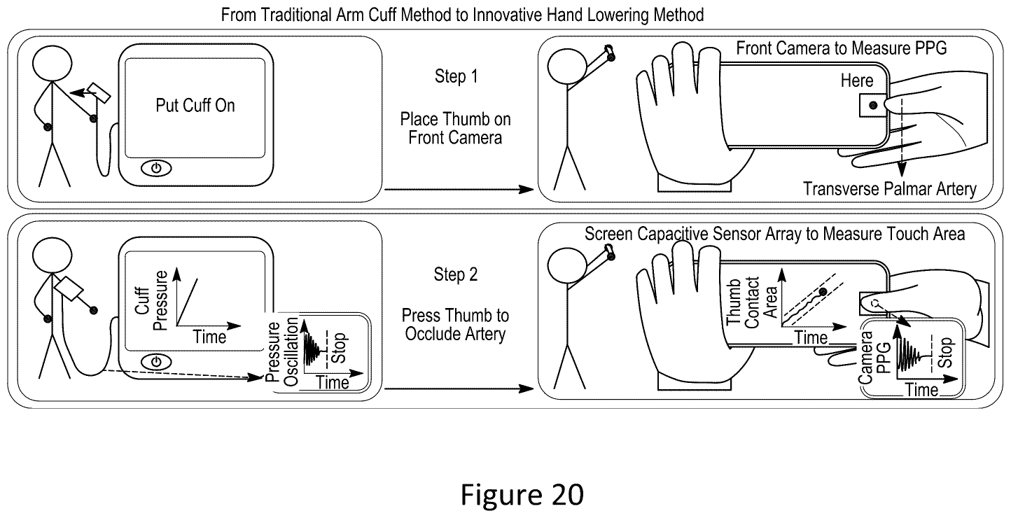

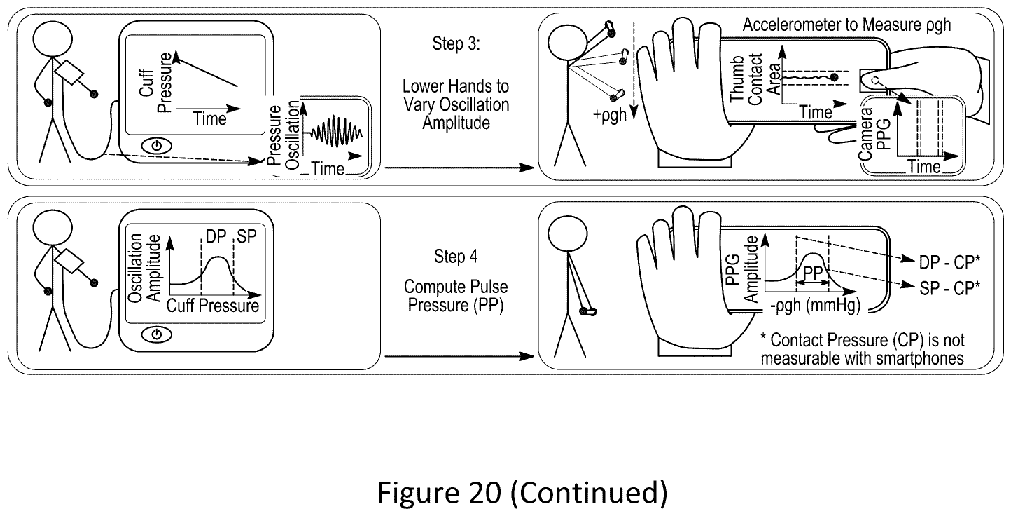

In one embodiment, a readily available device as known in the industry, such as but not limited to smartphones, tablets, laptops, watches and wearable devices, and the like, can be used to measure absolute PP as shown in FIG. 20. The steps parallel those used to make a traditional arm cuff BP measurement. Software that can be installed on any readily available device, such as through the operating system or through an app interface, can guide a user through body, finger and hand actuation phases to take the measurement. The app or software constructs the oscillogram and then uses it to compute PP. The software can instruct a user to raise his arm and hold the device, such as a phone, at an elevated height to start on the positive side of the transmural pressure regime where finger blood pressure is reduced. In Step 1, the devices guide a user to place their thumb on the front camera in a box that reflects their thumb size to measure PPG at the transverse palmar arch artery (the “artery”). In Step 2, the device guides a user with the screen contact area measurement to gradually press with the thumb to lower the transmural pressure until the artery is occluded (as measured via the PPG oscillations). The contact area at this point is recorded and used further for guiding the user to keep their contact area and, thus, contact pressure constant during a hand-lowering phase. During this phase, pgh is computed from the acceleration perpendicular to the screen and arm length. In Step 3, while maintaining the contact area, the device guides a user to lower their hands in increments (e.g., approximately 30 degrees or 20 degrees from +60 to −60 degrees relative to the heart for 3-5 s at a time) via audio/visual cues from the phone. In Step 4, PP is computed based on the PPG and pgh data collected during the hand lowering using an algorithm such as the one mentioned above. SP and DP cannot be computed because the thumb contact pressure is unknown. If the contact area goes out of some prespecified bounds too often, then the user is asked to try again.

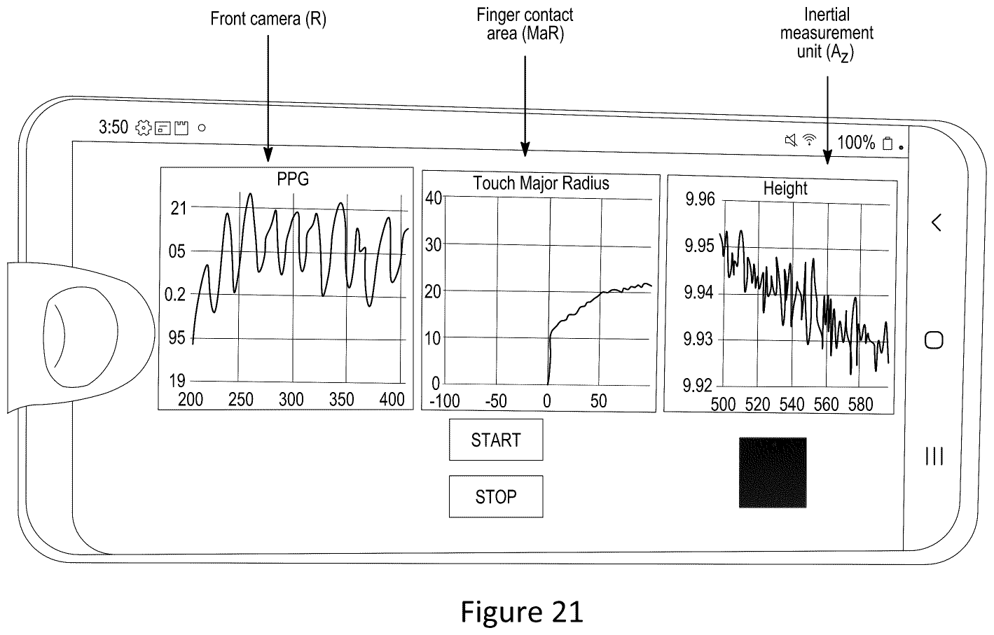

In one example, as shown in FIG. 21, an exemplary smartphone, a Samsung Galaxy S21, is provided that measures PPG (R, G, and B AC+DC channels), accelerometry (X, Y, and Z axes) and finger contact area (major and minor radius, x- and y-center position).

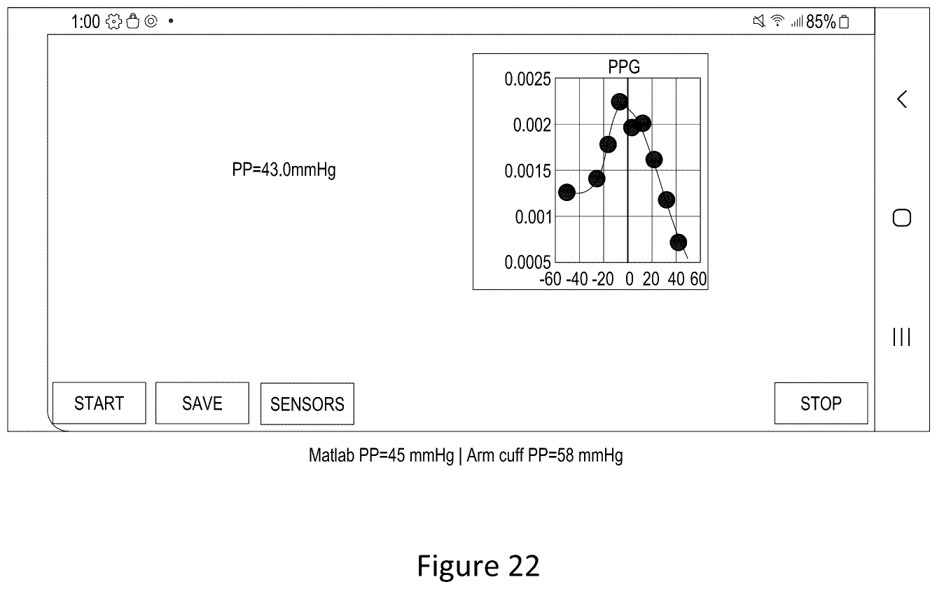

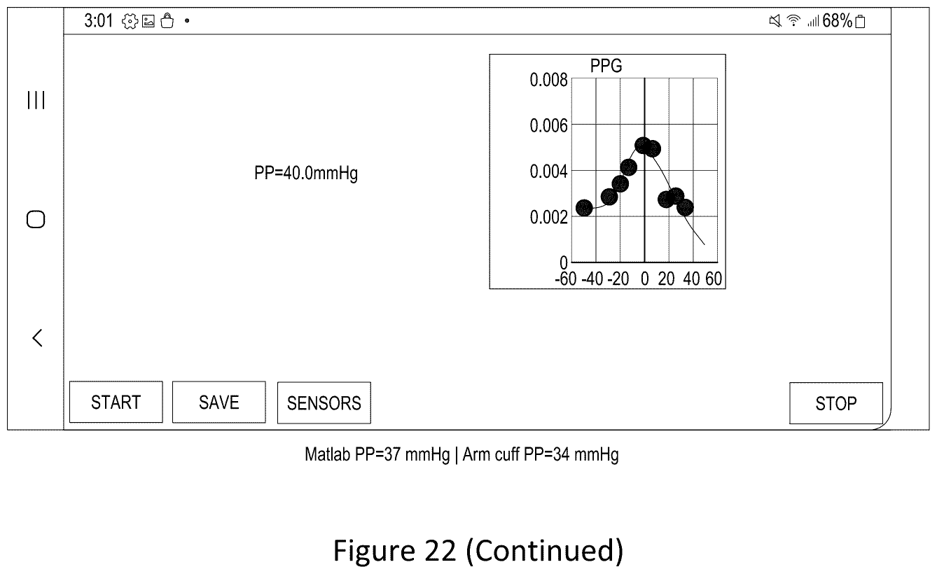

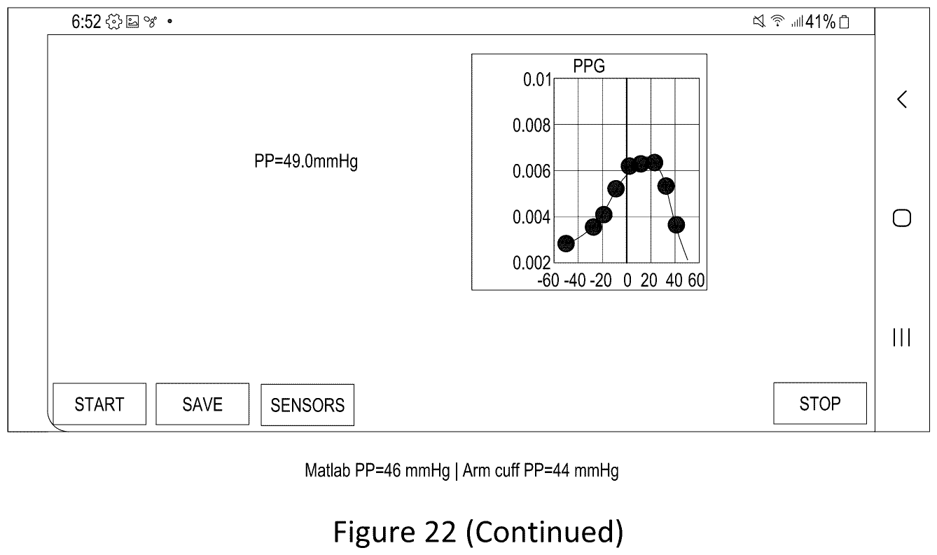

In one example, as shown in FIG. 22, exemplary measurements of PP are provided using the guidance system in FIG. 20 (N=3). The device computes an oscillogram based on 9 different heights (held at 5 seconds each on average).

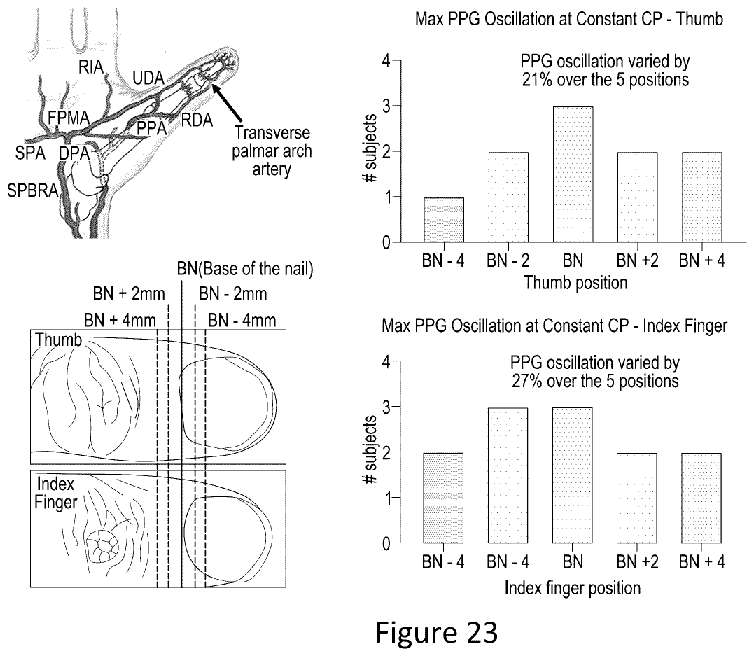

In one example, as shown in FIG. 23, the base of the fingernail can be positioned on top of the front camera to obtain large amplitude PPG oscillations. The optimal position could vary on the order of millimeters around the base of the nail.

For Step 1, the app can include a one-time initialization phase to measure PPG oscillations across different thumb positions and thereby identify the best thumb placement. This initialization can also create a profile of the thumb and its box dimensions for guiding the thumb placement thereafter, as shown at the top right of FIG. 20. An example of creating box dimensions is discussed in the article “An iPhone Application for Blood Pressure Monitoring via the Oscillometric Finger Pressing Method” by Chandrasekhar, A., Natarajan, K., Yavarimanesh, M. et al. Sci Rep 8, 13136 (2018). https://doi.org/10.1038/s41598-018-31632-x (also available at https://www.nature.com/articles/s41598-018-31632-x), the contents of which are incorporated by reference in its entirety.

For Step 2, thumb and hand actuation phases are used to measure BP with standard smartphones, and guidance through those phases is key to the ease of use of the app. This guidance can be done with the hand positioned at the heart level, lowered, or raised.

In certain embodiments, contact area (major & minor radii, centroid of touch) can be used for guidance. Parameters of contact area measured by the capacitive sensor array under the smartphone screen are used as a surrogate of applied contact pressure to guide the user to slowly increase the thumb pressure. The app measures the PPG oscillation amplitude in the background to determine the required pressure. For example, the PPG oscillation amplitude needs to be small when the hands are fully raised (i.e., occluded artery) and large when the hands are at heart level. The centroid of the touch could be used to correct for any non-obvious finger movements where the major or minor radii do not change. For ease of application, a mathematical transformation can be applied to the contact area measurement to approximately linearize the nonlinear contact area-contact pressure relationship.

In other embodiments, DC PPG can be used for guidance. DC PPG, as a function of the transmural pressure of an artery, is sigmoidal in shape and flattens at negative transmural pressure, as shown in FIG. 16. The user can be guided to increase pressure so that the DC PPG traces a sigmoid. The point where it flattens can be used as the required initial thumb pressure in the case that the hands are fully raised.

In other embodiments, AC PPG can be used for guidance. With increasing contact pressure, the PPG oscillations gradually increase in amplitude to reach a maximum and then decrease. When the hands are fully raised, the user can be guided to occlude the artery by pressing until the PPG oscillations appear and then vanish.

In other embodiments, applied finger pressure can be used for guidance. For phones or devices that have 3D touch, the thumb force measured by the strain gauge array under the screen can be used to guide the arterial occlusion in the case that the hands are fully raised. The app measures PPG oscillation amplitude in the background to determine the required thumb pressure.

For Step 3, the user needs to maintain their thumb pressure and lower/raise their hands continuously or in increments.

In one embodiment, contact area parameters (major & minor radii, centroid of touch) can be used for guiding the hand raising/lowering in Step 3. A plot of the contact area (via any possible combination of the touch parameters) versus time is displayed. The user is then guided to maintain the contact area throughout the hand-lowering/raising procedure. Audio/color cues are given to the user to guide them to lower/raise their hands in increments.

In another embodiment, the accelerometer can be used for guidance. Pgh is measured with the user's arm length and the accelerometer via a single axis or application of principal components analysis to all axes. The latter can allow the user to hold the phone at arbitrary orientations. A plot of pgh versus time then guides the user to continuously lower/raise their hands over a 20-40 sec period. In particular, the guidance is such that pgh changes linearly over the time period. No guidance is given to maintain the thumb pressure during the hand lowering/raising. However, contact area variations and non-physiologic oscillogram measurements in the background could be used to prompt the user to try again. Furthermore, the user can be taught to maintain the thumb contact pressure in the initialization phase. In this phase, the user would practice maintaining AC or DC PPG over time while the hands are at different but fixed levels relative to the heart.

In other embodiments, applied finger pressure can be used for guidance. For phones with 3D touch, the user is guided to maintain the thumb contact pressure via the screen force measurement. The user is guided to lower/raise the hands via audio/color cues.

In other embodiments, DC PPG can be used for guidance. A plot of DC PPG versus time is displayed. The user is guided to lower/raise their hands while maintaining the thumb contact pressure by producing a sigmoidal DC PPG over 20-40 sec.

Using different means of guidance, multiple versions of the app are possible and could be tailored to the user's preference. The app can include a video demonstration to teach the user how to perform the procedure.

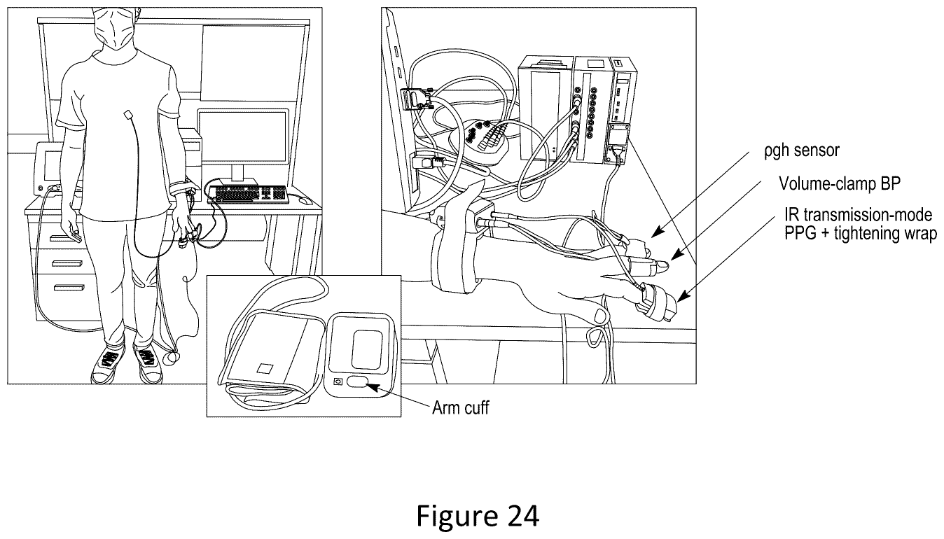

In one example, as shown in FIG. 24, standard wired sensors including a pgh sensor can be used for testing a hand-raising protocol. The pgh sensor can be a tube with fluid interfaced to a pressure transducer.

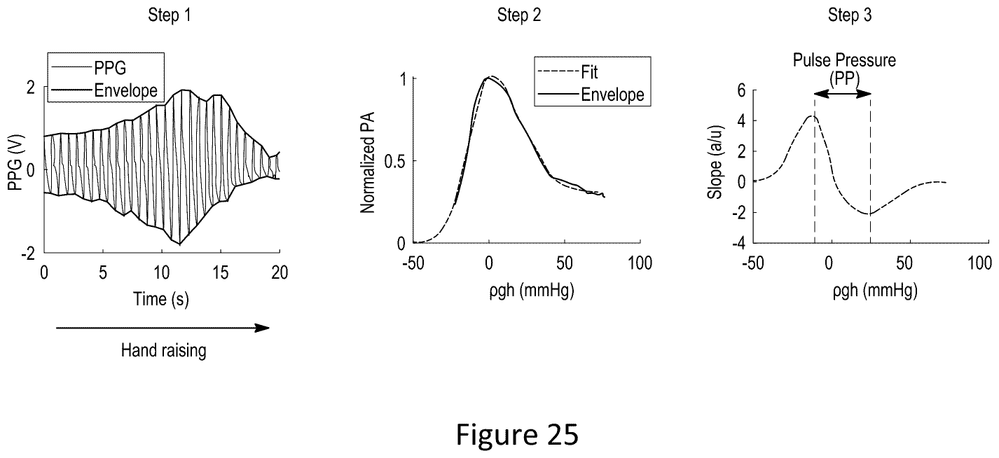

In one example, as shown in FIG. 25, a three-step procedure can be used to compute PP. First, the upper and lower envelopes of the PPG oscillations are detected. Second, the oscillogram is constructed by plotting the difference in the envelopes as a function of pgh. Then, two half-Gaussian functions are fitted to the normalized oscillogram. Third, the width of the oscillogram is computed as the change in pgh between the maximum and the minimum slopes of the fit. This width is used as an index of PP.

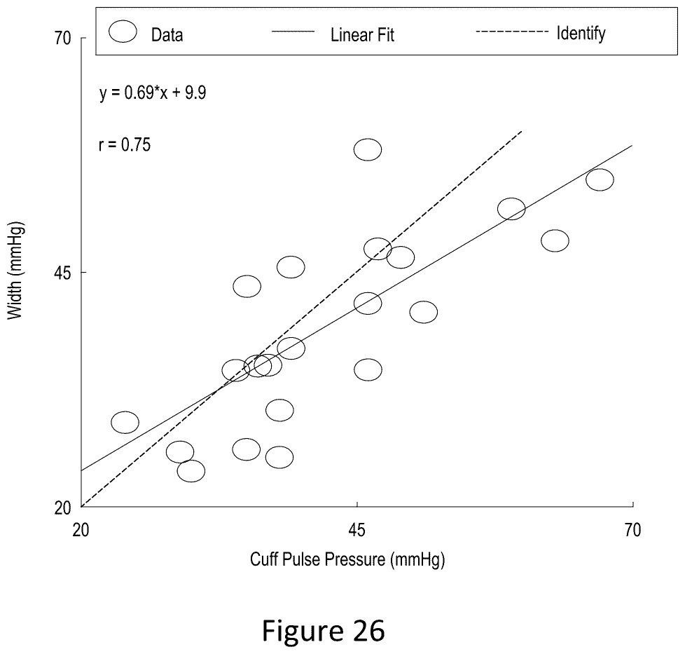

As shown in FIG. 26, the width of the oscillogram is representative of PP. Further accuracy improvement can be obtained by converting finger PP to arm PP (e.g., using the method in FIG. 19, where the maximum oscillation is scaled by the width of the oscillogram after removing its mean value). The mean and standard deviation of the PP errors were −2.9 and 7.4 mmHg.

As shown in FIG. 26, the oscillometric hand raising/lowering method can work using any kind of sensor known in the field. In certain embodiments, this includes ideal sensors such as those in FIG. 24 to develop the PP computation algorithm. Using software such as a readily available device app for algorithm development, by contrast, can introduce random variability in the data but can be utilized if ideal sensors are unavailable.

In other embodiments, estimating DP includes measuring the maximum thumb contact area by pressing the thumb firmly. If the contact area from the initial press (Step 2 of FIG. 20) is relatively small compared to the maximum contact area, then DP can be on the lower side. Thus, for people <65 years, a high PP is not a sign of risk.

In certain embodiments, estimating thumb contact pressure can include using available software to capture the thumb contact pressure, such as but not limited to the software provided with the University of Michigan force phone app, as discussed in the article “Force-feeling phone: Software lets mobile devices sense pressure” (available at https://news.umich.edu/force-feeling-phone-software-lets-mobile-devices-sense-pressure/). In another embodiment, estimating thumb contact pressure includes using deep touch technology such as Google Deep Touch as described in the article “Sensing Force-Based Gestures on the Pixel 4” (available at https://ai.googleblog.com/2020/06/sensing-force-based-gestures-on-pixel-4.html).

### Example 2

### Models for Improving Oscillometric BP Computation

The disclosed subject matter further provides reliable BP computation that can be used for the clinical BP range. For example, the disclosed subject matter provides a model for improving oscillometric BP computation.

The disclosed model can find BP signatures in finger oscillometric measurements. For example, the disclosed model can be the sigmoidal blood volume-pressure relationship of arteries. The input to the model can be the BP waveform at different external pressures, and the output can be the varying blood volume waveform.

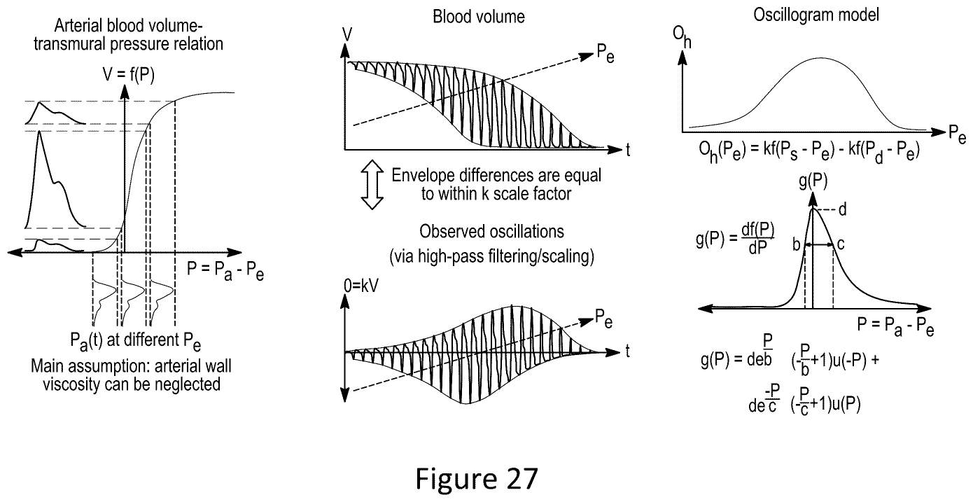

FIG. 27 shows the model-predicted blood volume waveform in response to an external pressure ramp due to finger pressing. High-pass filtering and scaling of this waveform predicts the PPG oscillation waveform that is measured. Comparing the envelopes of the two waveforms indicates that the oscillogram, which is again the oscillation height versus external pressure function, is proportional to the difference in the y-axis reversed sigmoidal relationships evaluated at SP and DP. The derivative of the sigmoidal relationship is the arterial compliance curve. This curve can be well represented with a three-parameter exponential-linear function, which completes the model.

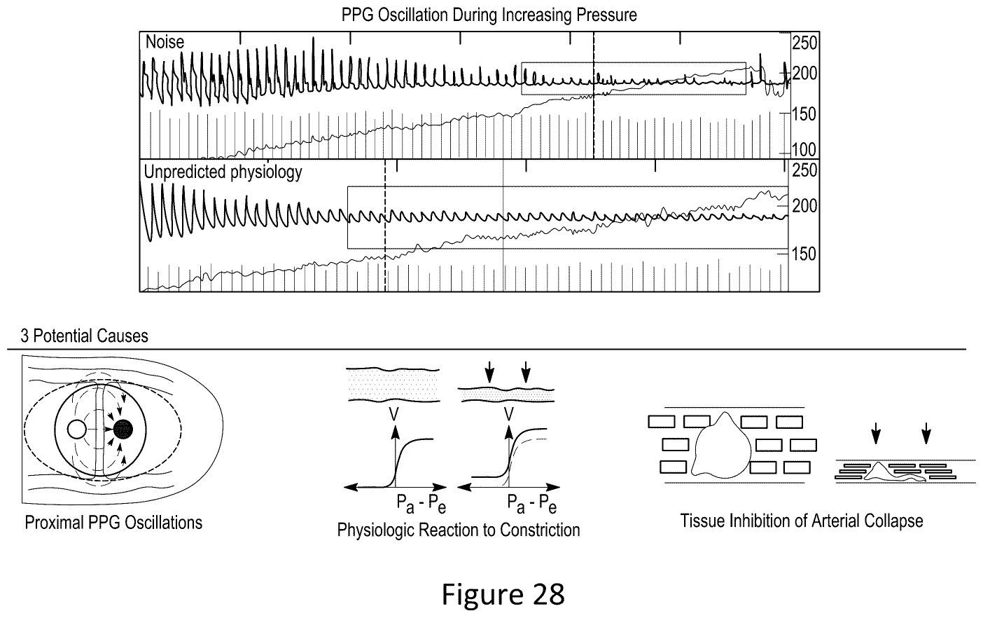

In FIG. 28, PPG oscillation during increasing pressure is shown. In certain embodiments, zero oscillation can include noise floor or unchanging oscillations. The causes of the noise or the unpredicted physiology can include proximal PPG oscillations, physiologic reaction to constriction, and tissue inhibition of arterial collapse.

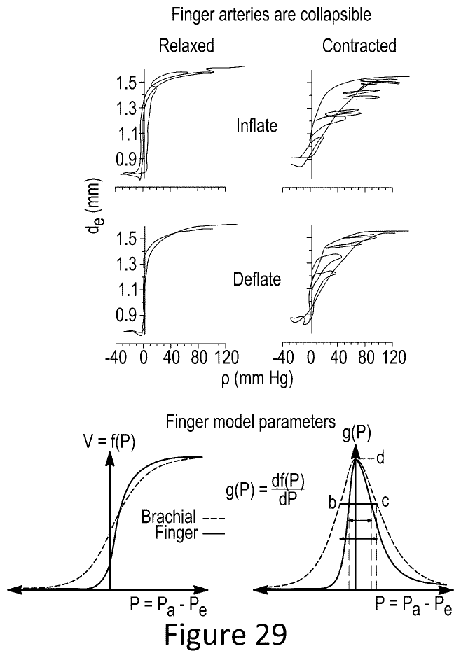

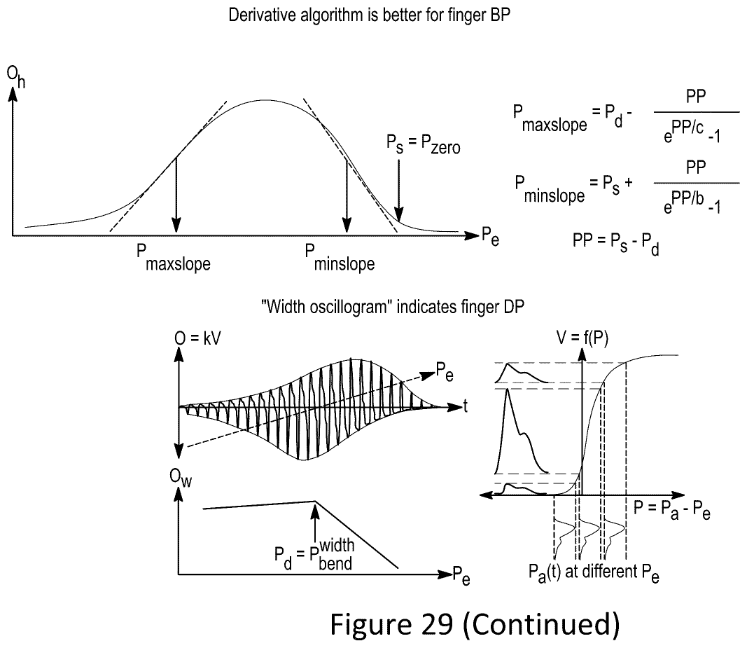

Finger arteries can become fully occluded or collapse when the external pressure is just slightly above BP. As shown in FIG. 29, this property indicates that the b model parameter, which denotes the left width of the arterial compliance curve, is smaller for finger arteries than the traditional brachial artery. Additional data suggest that the c parameter, which denotes the right width of the curve, is also smaller for finger arteries. So, the disclosed model can predict that the external pressures at the maximum and minimum slopes of the oscillogram can better indicate finger DP and SP than brachial DP and SP. However, differentiation can amplify noise.

The model also indicates that the oscillation width decreases with increasing external pressure. Because of the small b parameter value, the width reduction begins once the external pressure exceeds DP. Hence, the model specifically predicts that an unconventional “width” oscillogram denotes DP via a “bend” in the curve. The model with small b further indicates that when the external pressure starts to exceed SP, the oscillations can be entirely abolished. However, detecting arterial occlusion turns out to be difficult for noise and other reasons, as mentioned above.

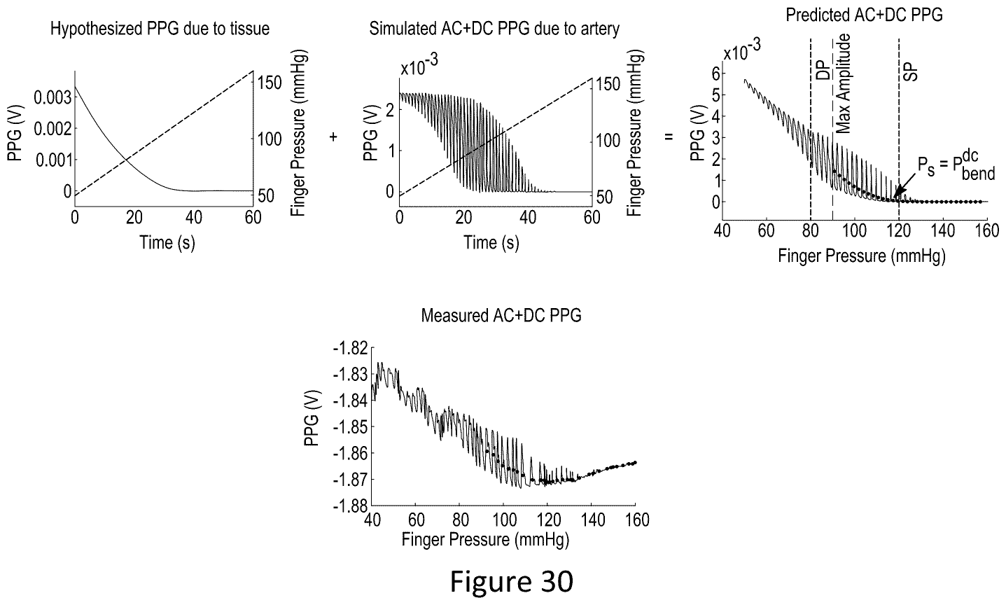

As shown in FIG. 30, the disclosed model, based on the Beer-Lambert Law, can be further extended to AC+DC PPG, assuming that tissue compression due to arterial expansion is small compared to tissue compression due to applied finger pressure, and that tissue is fully compressed between DP and SP. As the DC component of PPG can help in robustly detecting arterial occlusion and thus SP computation, the model can be extended to AC+DC PPG and prediction. With a slow external pressure ramp due to finger pressing, the tissue compresses up to a certain pressure until it is fully compressed. So, PPG decreases and then saturates due to tissue. FIG. 30 shows the simulated AC+DC PPG due to the artery from the original model. Summing the two can provide the predicted AC+DC PPG. The extended model can predict that AC+DC PPG versus finger pressure denotes SP via a bend (i.e., a sudden change in the slope of the function) in the curve. The model prediction can provide accuracy and similarity to the actual measurement by using the average PPG beat.

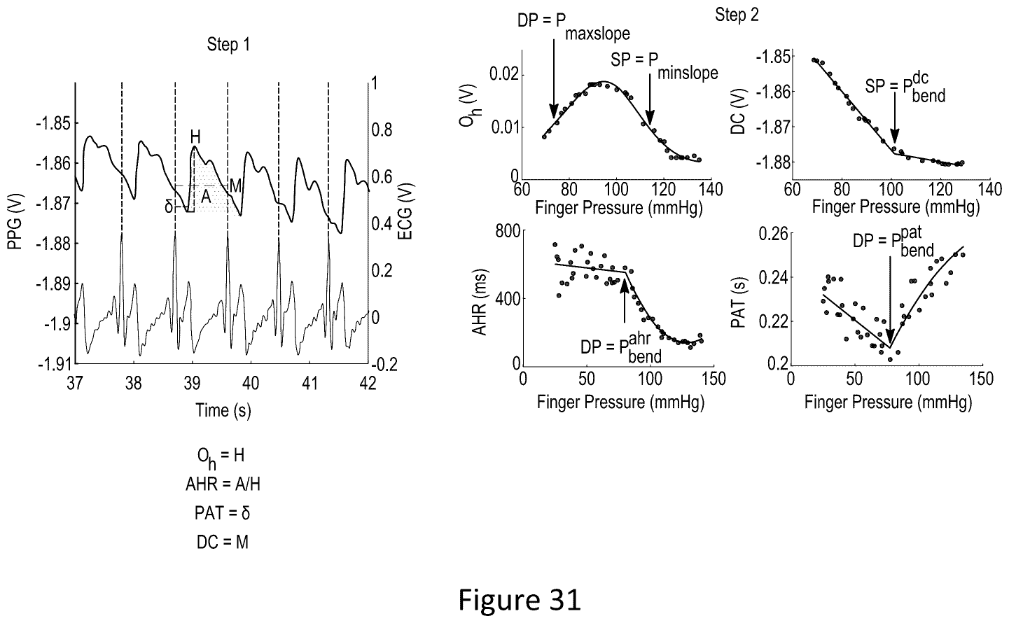

In certain embodiments, the disclosed subject matter provides BP computation algorithms based on the disclosed model predictions. FIG. 31 shows examples of steps for the model-based DP and SP computation algorithms. During the first step, oscillation width can be quantified using the area-to-height ratio and via pulse arrival time obtained with an ECG, and the AC+DC PPG can be quantified via the mean over each ECG RR interval (e.g., average PPG beat). During the second step, these three features and the oscillation height are plotted against finger pressure. The model-predicted signatures can be detected to compute three DP features and two SP features.

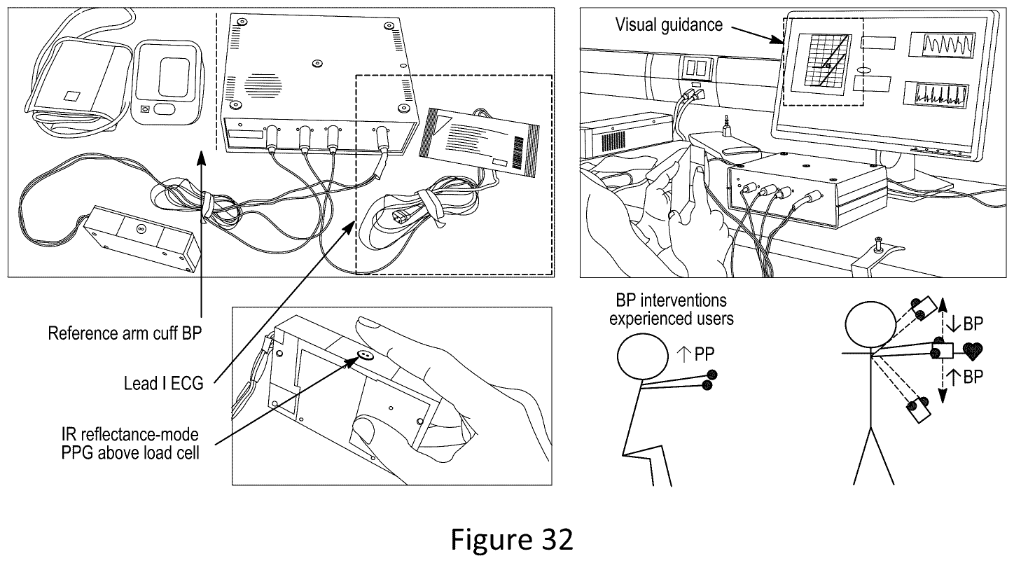

FIG. 32 shows images illustrating an example benchtop system for assessing the disclosed device and models. The benchtop system can be used for evaluating the disclosed algorithms against an arm cuff device in patients. A user can also perform interventions to vary their BP.

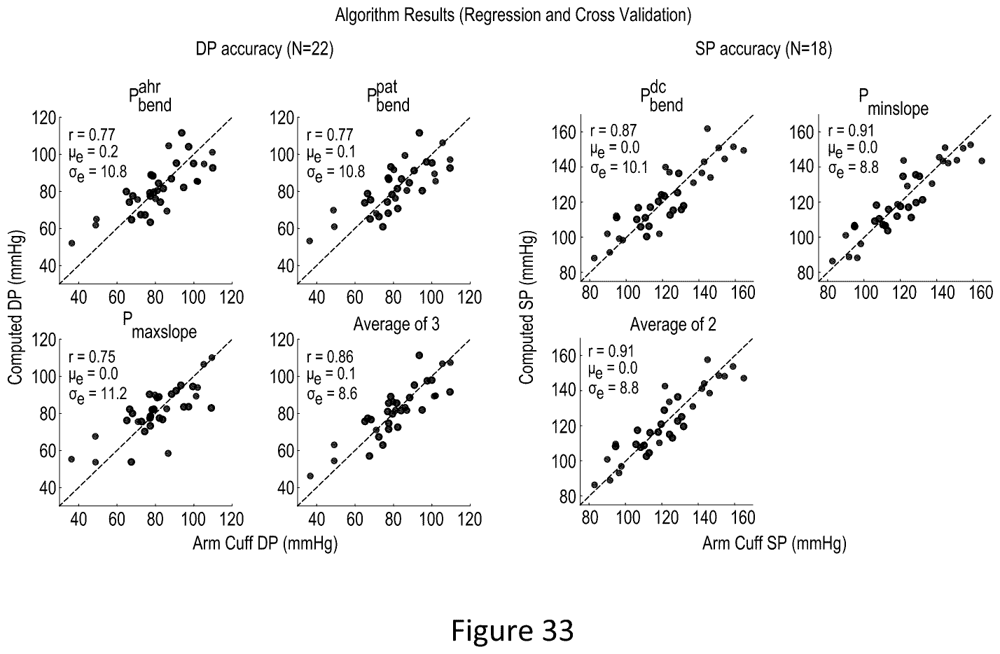

FIG. 33 are graphs illustrating the accuracy of the disclosed models and algorithms. The average of the three DP features and the average of the two SP features yielded BP errors of around 8.5 mmHg and correlations of about 0.9 with respect to the automatic cuff device. The width oscillation variations helped in the DP computation, whereas the DC PPG did not improve the SP computation over the PPG oscillations. However, in contrast to PPG oscillations, DC PPG typically shows a clear fiducial marker around SP and could ultimately prove beneficial.

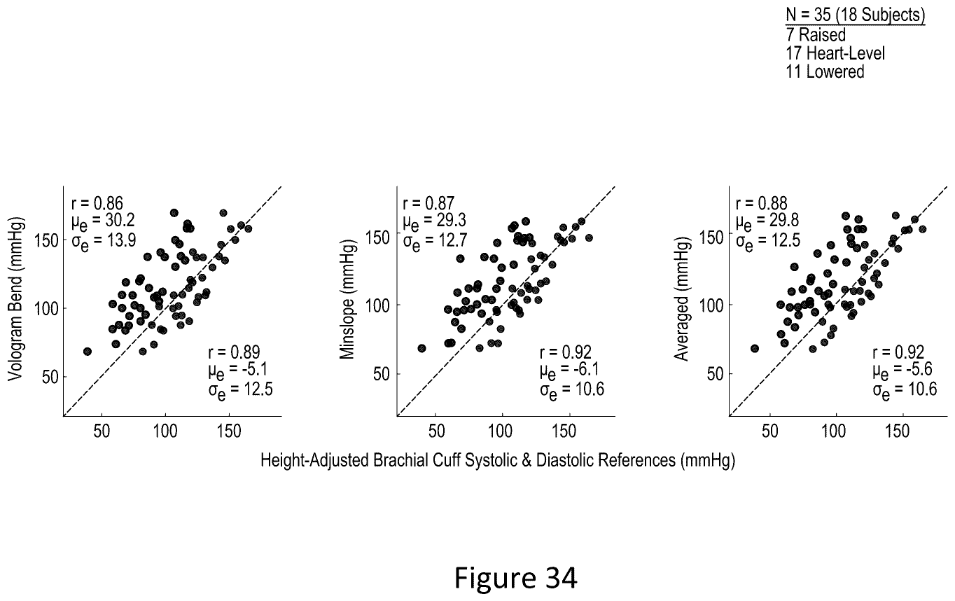

FIG. 34 shows the correlation between systolic and diastolic references in DC PPG. A total of 35 measurements were taken at various arm positions (i.e., raised, heart-level, and lowered) across 18 subjects. Out of the 35 measurements, 7 were taken at raised levels, 17 were taken at heart-level, and 11 were taken at lowered levels. FIG. 34 shows high-adjusted brachial cuff systolic and diastolic references.

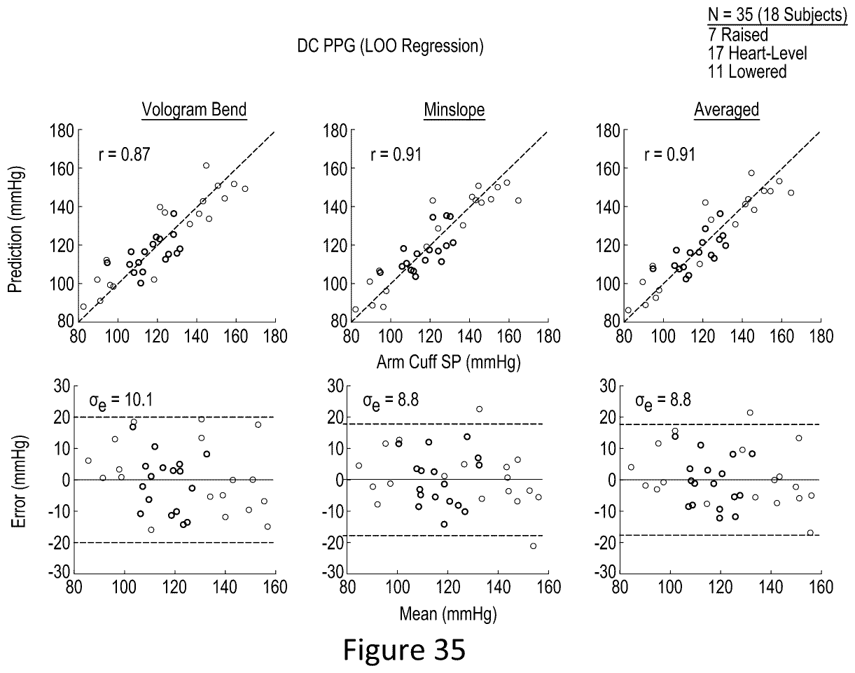

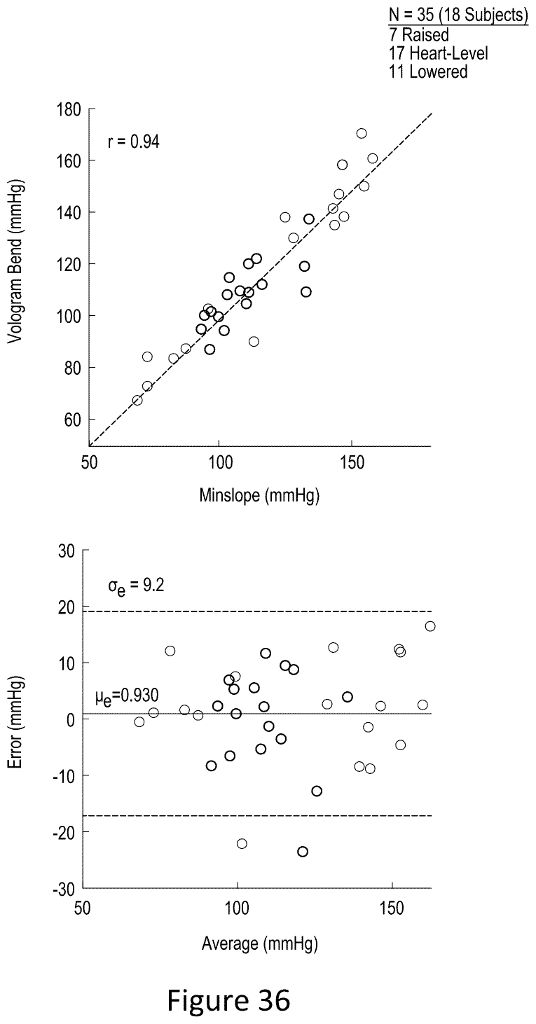

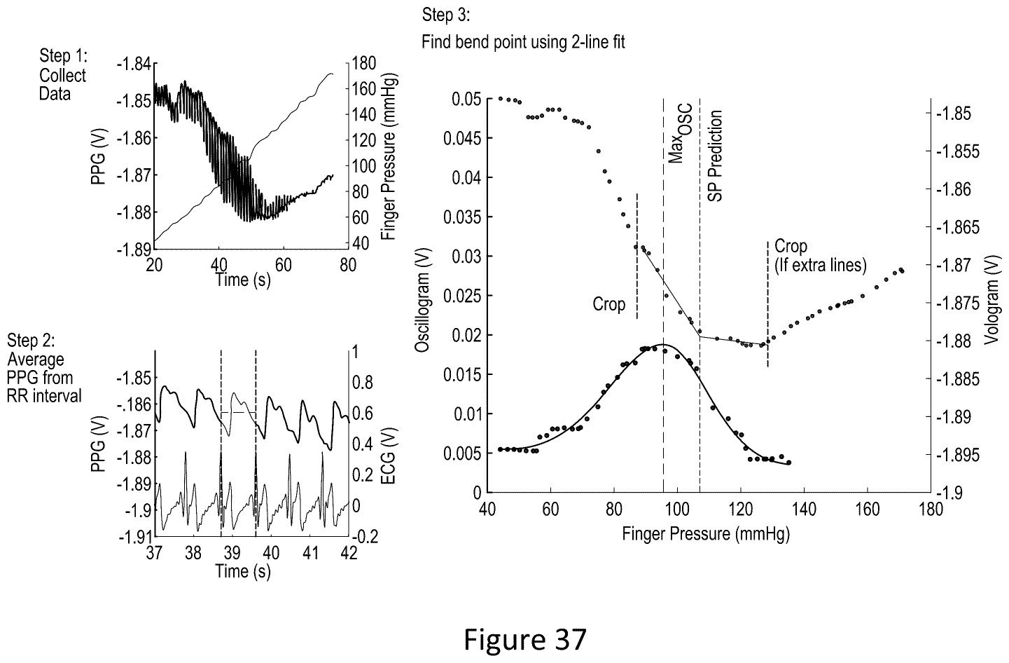

In FIG. 35, DC PPG results using leave-one-out (LOO) regression are shown. A total of 35 measurements were taken at various arm positions (i.e., raised, heart-level, and lowered) across 18 subjects. Out of the 35 measurements, 7 were taken at raised levels, 17 were taken at heart-level, and 11 were taken at lowered levels. FIG. 36 shows a correlation between the vologram bend measurement and the minslope prediction. FIG. 37 shows an example method for the vologram bend measurement. As shown in FIG. 37, the method can include collecting data, averaging PPG from RR interval, and finding a bend point using a 2-line fit.

As shown in FIGS. 33-36, the regressed Minslope prediction of SP (correlation coefficient r, standard deviation of error σₑ) improved with the disclosed measurement techniques (e.g., from the AC study (r: 0.69. σₑ: 13.1) to the DC study (r 0.91, σₑ: 8.8)). This can be due to the improved measurement protocol (i.e., cuff placement, required press linearity, and defined measurement-device orientations), the accuracy of height offsets (i.e., height probe and robust heart level definition), or enhanced analysis (i.e., removed pulses recorded during non-increasing pressing, required per-subject consistency of data).

The Vologram bend and Minslope come from different PPG information (i.e., shape and amplitude), predict SP comparably on average, and can have little margin for improvement due to the branchial arm cuff reference.

Volograms (AC & DC PPG vs finger pressure) show a clear fiducial marker that can be indicative of SP and can be reproducible. As such, the analysis of high-noise data can differentiate DC PPG as a more noise-robust predictor of SP.

While the disclosed subject matter is described herein in terms of certain preferred embodiments, those skilled in the art will recognize that various modifications and improvements can be made to the disclosed subject matter without departing from the scope thereof. Additional features known in the art likewise can be incorporated, such as disclosed in WO Publication WO2013003787A2, U.S. Provisional Application No. 63/135,430, and U.S. Provisional Application No. 63/388,021, the contents of each of which are incorporated by reference in their entireties. Moreover, although individual features of one embodiment of the disclosed subject matter can be discussed herein or shown in the drawings of the one embodiment and not in other embodiments, it should be apparent that individual features of one embodiment can be combined with one or more features of another embodiment or features from a plurality of embodiments.

In addition to the various embodiments depicted and claimed, the disclosed subject matter is also directed to other embodiments having any other possible combination of the features disclosed and claimed herein. As such, the particular features presented herein can be combined with each other in other manners within the scope of the disclosed subject matter such that the disclosed subject matter includes any suitable combination of the features disclosed herein. Thus, the foregoing description of specific embodiments of the disclosed subject matter has been presented for purposes of illustration and description. It is not intended to be exhaustive or to limit the disclosed subject matter to those embodiments disclosed. It will be apparent to those skilled in the art that various modifications and variations can be made in the device, method and system of the disclosed subject matter without departing from the spirit or scope of the disclosed subject matter. Thus, it is intended that the disclosed subject matter include modifications and variations that are within the scope of the appended claims and their equivalents.

What is claimed is:

1. A method for determining diastolic blood pressure of a user using a device with a photoplethysmography (PPG)-force sensor unit comprising:
providing visual or audio instructions to the user with the device, wherein the instructions instruct the user to position a finger on the PPG-force sensor unit and to press the finger on the PPG-force sensor unit at varying finger pressures;
measuring PPG oscillations of the finger and the finger pressures with the PPG-force sensor unit;
computing a width of each of the PPG oscillations as a function of the finger pressure;
computing diastolic blood pressure using the PPG oscillation width versus finger pressure function; and
outputting the diastolic blood pressure on a graphical user interface of the device or sending the diastolic blood pressure to a database repository.

2. The method of claim 1, further comprising detecting a bend in the PPG oscillation width to the finger pressure function.

3. The method of claim 2, wherein the bend in the PPG oscillation width to the finger pressure function is detected by fitting at least two curves to the function and using an intersection of the curves.

4. The method of claim 1, wherein the width of each PPG oscillation is computed as an area-to-height ratio of the oscillation.

5. The method of claim 1, further comprising:
measuring an electrocardiogram (ECG) with additional electrodes incorporated into the device; and
computing the width of each PPG oscillation as a pulse arrival time for each of the oscillations detected as a time delay between an R-wave of the ECG and a PPG foot.

6. The method of claim 5, further comprising determining systolic blood pressure using a value of the pulse arrival time at a bend of the pulse arrival time to the finger pressure function.

7. The method of claim 1, wherein the diastolic blood pressure is computed using additional features extracted from the PPG oscillations and finger pressure.

8. The method of claim 7, wherein the additional features include a finger pressure at a maximum slope of the PPG oscillation amplitude to finger pressure function.

9. A method for determining systolic blood pressure of a user using a device with a photoplethysmography (PPG)-force sensor unit and electrocardiogram (ECG) electrodes comprising:
providing visual or audio instructions to the user with the device, wherein the instructions instruct the user to position a finger on the PPG-force sensor unit and to press the finger on the PPG-force sensor unit at varying finger pressures;
measuring a total PPG including a direct current (DC) component, PPG oscillations, and the applied finger pressure with the PPG-force sensor unit;
measuring ECG with the electrodes;
computing an average of each PPG beat over the R-wave to R-wave interval of the ECG as a function of the applied finger pressure; and
computing the systolic blood pressure using the PPG average to the finger pressure function.

10. The method of claim 9, further comprising detecting a bend in the PPG average to the finger pressure function.

11. The method of claim 10, wherein the bend in the PPG average to the finger pressure function is detected by fitting at least two curves to the function and using an intersection of the curves.

12. The method of claim 9, wherein systolic blood pressure is computed using additional features extracted from the total PPG, finger pressure, and ECG.

13. The method of claim 12, wherein the additional features include a finger pressure at a minimum slope of the PPG oscillation amplitude to finger pressure function.

14. The method of claim 12, wherein the additional features include a value of the pulse arrival time at the bend of the pulse arrival time to the finger pressure function.

15. A method for determining pulse pressure of a user using a device comprising:
providing visual or audio instructions to a user with the device, wherein the instructions instruct the user to position a finger on a camera and a screen of the device to measure a total photoplethysmography (PPG) from the finger via the camera of the device and finger contact parameters via a touch screen sensor of the screen;
providing visual or audio instructions to the user with the device to apply a finger pressure on the camera and the screen based on the measurements;
providing visual or audio instructions to the user with the device to lower or raise a hand of the finger with respect to a heart level of the user while maintaining the finger contact parameters;
measuring a hydrostatic blood pressure change (pgh) in the finger by using an accelerometer measurement of the device and an arm length; and
computing pulse pressure from the PPG and pgh measurements.

16. The method of claim 15, further comprising providing an instruction with the device for a one-time initialization to determine an optimal placement guide for the finger on the camera and the screen.

17. The method of claim 15, wherein the finger contact parameters comprises a touch centroid.

18. The method of claim 15, wherein the PPG oscillations are used as a guide to determine the amount of finger pressure on the screen and the camera to be maintained during hand raising or lowering.

19. The method of claim 15, wherein the PPG oscillations and finger contact parameters are used as a guide to determine the amount of finger pressure on the screen and the camera to be maintained during hand raising or lowering.

20. The method of claim 15, where finger contact parameters are used as the guide to maintain the finger pressure on the screen and the camera during the hand raising or lowering.

21. The method of claim 15, wherein a timer is used as a further guide to indicate the amount of time to take for hand raising or lowering.

22. The method of claim 15, wherein pulse pressure is computed using at least one of pgh at minimum and maximum slopes of the PPG oscillation amplitude to pgh function or a of the PPG oscillation amplitude as a function of pgh.

23. The method of claim 15, further comprising measuring diastolic blood pressure by measuring a maximum finger contact parameter via firm finger pressing by the user and comparing a finger contact parameter with a maximal finger contact parameter to determine if diastolic blood pressure is low.

24. A method for determining blood pressure using a device with a photoplethysmography (PPG)-force sensor unit and ECG electrodes comprising:
receiving measurements from a touch of a finger on the PPG-force sensor unit;
receiving ECG from the electrodes;
computing diastolic blood pressure with a processor of the device using a width of each alternative current (AC) blood volume oscillation versus finger pressure function;
computing by the processor systolic blood pressure using an average of each direct current (DC) blood volume beat over the RR interval of the ECG versus finger pressure function; and
outputting the blood pressure on a graphical user interface of the device or sending the blood pressure to a database repository.

25. A system for determining blood pressure of a subject comprising
sensors configured to measure finger pressure, a finger photoplethysmography (PPG) oscillation, a finger PPG DC component, and ECG;
a display configured to provide visual or audio instructions to instruct the subject to position a finger at a predetermined location; and
a processor configured to:
compute diastolic blood pressure and systolic blood pressure, wherein the diastolic blood pressure is calculated using a PPG oscillation width versus finger pressure function, and wherein the systolic blood pressure is calculated using a PPG average; and
display the computed diastolic blood pressure and the systolic blood pressure on the display.
